(19) United States
(12) Patent Application Publication
(10) Pub. No.: US 2003/0148970 A1
(43) Pub. Date: Aug. 7, 2003

(54) Methods for specifically inhibiting histone deacetylase-4

(76) Inventors: Jeffrey M. Besterman, Baie D-urfe (CA); Claire Bonfils, Montreal (CA); Zuomei Li, Kirkland (CA); Soon Hyung Woo, Beaconsfield (CA); Arkadii Vaisburg, Kirkland (CA); Daniel Delorme, St.-Lazare (CA); Marielle Fournel, LaSalle (CA); Rico Lavoie, Hamden, CT (US)

Correspondence Address: KEOWN & ASSOCIATES, 500 WEST CUMMINGS PARK, SUITE 1200, WOBURN, MA, 01801, US

(21) Appl. No.: 10/052,390

(22) Filed: Jan. 14, 2002

Related U.S. Application Data
(60) Provisional application No. 60/261,522, filed on Jan. 12, 2001.

(51) Int. Cl. A61K 48/00; C12N 5/02

(52) U.S. Cl. 514/44; 435/375

(57) ABSTRACT

This invention relates to the inhibition of histone deacetylase (HDAC) expression and enzymatic activity. The invention provides methods and reagents for inhibiting HDAC-4 and HDAC-1 by inhibiting expression at the nucleic acid level or inhibiting enzymatic activity at the protein level.

### BACKGROUND OF THE INVENTION

1. Field of the Invention

The present invention relates to the fields of molecular biology and medicine. More specifically, the invention relates to the fields of gene expression and oncology.

2. Summary of the Related Art

Chromatin is the complex of proteins and DNA in the nucleus of eukaryotes. Chromatin proteins provide structural and functional organization to nuclear DNA. The nucleosome is the fundamental unit of structural organization of chromatin. The nucleosome principally consists of (1) the core histones, termed H2A, H2B, H3, and H4, which associate to form a protein core particle, and (2) the approximately 146 base pairs of DNA wrapped around the histone core particle. The physical interaction between the core histone particle and DNA principally occurs through the negatively charged phosphate groups of the DNA and the basic amino acid moieties of the histone proteins. (Csordas, Biochem. J., 286:23-38 (1990)) teaches that histones are subject to posttranslational acetylation of their epsilon-amino groups of N-terminal lysine residues, a reaction that is catalyzed by histone acetyl transferase (HAT). The posttranslational acetylation of histones has both structural and functional, i.e., gene regulatory, consequences.

Acetylation neutralizes the positive charge of the epsilon-amino groups of N-terminal lysine residues, thereby influencing the interaction of DNA with the histone core particle of the nucleosome. Thus, histone acetylation and histone deacetylation (HDAC) are thought to impact chromatin structure and gene regulation. For example, Taunton et al., Science, 272:408-411 (1996), teaches that access of transcription factors to chromatin templates is enhanced by histone hyperacetylation. Taunton et al. further teaches that an enrichment in underacetylated histone H4 has been found in transcriptionally silent regions of the genome.

Studies utilizing known HDAC inhibitors have established a link between acetylation and gene expression. Yoshida et al, Cancer Res. 47:3688-3691 (1987) discloses that (R)-Trichostatin A (TSA) is a potent inducer of differentiation in murine erythroleukemia cells. Yoshida et al., J. Biol. Chem. 265:17174-17179 (1990) teaches that TSA is a potent inhibitor of mammalian HDAC.

Numerous studies have examined the relationship between HDAC and gene expression. Taunton et al., Science 272:408-411 (1996), discloses a human HDAC that is related to a yeast transcriptional regulator. Cress et al., J. Cell. Phys. 184:1-16 (2000), discloses that, in the context of human cancer, the role of HDAC is as a corepressor of transcription. Ng et al., TIBS 25:March (2000), discloses HDAC as a pervasive feature of transcriptional repressor systems. Magnaghi-Jaulin et al., Prog. Cell Cycle Res. 4:41-47 (2000), discloses HDAC as a transcriptional co-regulator important for cell cycle progression.

The molecular cloning of gene sequences encoding proteins with HDAC activity has established the existence of a set of discrete HDAC enzyme isoforms. Grozinger et al., Proc. Natl. Acad. Sci. USA, 96:4868-4873 (1999), teaches that HDACs may be divided into two classes, the first represented by yeast Rpd3-like proteins, and the second represented by yeast Hda1-like proteins. Grozinger et al. also teaches that the human HDAC-1, HDAC-2, and HDAC-3 proteins are members of the first class of HDACs, and discloses new proteins, named HDAC-4, HDAC-5, and HDAC-6, which are members of the second class of HDACs. Kao et al., Gene & Development 14:55-66 (2000), discloses an additional member of this second class, called HDAC-7. More recently, Hu, E. et al. J. Bio. Chem. 275:15254-13264 (2000) discloses the newest member of the first class of histone deacetylases, HDAC-8. It has been unclear what roles these individual HDAC enzymes play.

Known inhibitors of mammalian HDAC have been used to probe the role of HDAC in gene regulation for some time. Yoshida et al., J. Biol. Chem. 265:17174-17179 (1990) discloses that (R)-Trichostatin A (TSA) is a potent inhibitor of mammalian HDAC. Yoshida et al, Cancer Res. 47:3688-3691 (1987) discloses that TSA is a potent inducer of differentiation in murine erythroleukemia cells.

Known inhibitors of histone deacetylase are all small molecules that inhibit histone deacetylase activity at the protein level. Moreover, all of the known histone deacetylase inhibitors are non-specific for a particular histone deacetylase isoform, and more or less inhibit all members of both the histone deacetylase families equally. (Grozinger, C. M., et al., Proc. Natl. Acad. Sci. U.S.A. 96:4868-4873 (1999)). For example, see Marks et al., J. National Cancer Inst. 92:1210-1216 (2000), which reviews histone deacetylase inhibitors and their role in studying differentiation and apoptosis.

Therefore, there remains a need to develop reagents for inhibiting specific histone deacetylase isoforms. There is also a need for the development of methods for using these reagents to modulate the activity of specific histone deacetylase isoforms and to identify those isoforms involved in tumorigenesis and other proliferative diseases and disorders.

### BRIEF SUMMARY OF THE INVENTION

The invention provides methods and reagents for modulating the activity of histone deacetylase (HDAC) isoforms. For example, the invention provides methods and reagents for inhibiting HCAC isoforms, particularly HDAC-1 and HDAC-4, by inhibiting expression at the nucleic acid level or enzymatic activity at the protein level. The invention provides for the specific inhibition of specific histone deacetylase isoforms involved in tumorigenesis and thus provides a treatment for cancer. The invention further provides for the specific inhibition of particular HDAC isoforms involved in cell proliferation, and thus provides a treatment for cell proliferative diseases and disorders.

The inventors have made the surprising discovery that the specific inhibition of HDAC-4 dramatically induces apoptosis and growth arrest in cancerous cells. Accordingly, in a first aspect, the invention provides agents that inhibit the activity of the HDAC-4 isoform.

In certain preferred embodiments of the first aspect of the invention, the agent that inhibits the HDAC-4 isoform is an oligonucleotide that inhibits expression of a nucleic acid molecule encoding the HDAC-4 isoform. The nucleic acid molecule encoding the HDAC-4 isoform may be genomic DNA (e.g., a gene), cDNA, or RNA. In some embodiments, the oligonucleotide inhibits transcription of mRNA encoding the HDAC-4 isoform. In other embodiments, the oligonucleotide inhibits translation of the HDAC-4 isoform. In certain embodiments the oligonucleotide causes the degradation of the nucleic acid molecule.

In a preferred embodiment thereof, the agent of the first aspect of the invention is an antisense oligonucleotide complementary to a region of RNA that encodes a portion of HDAC-4 or to a region of double-stranded DNA that encodes a portion of HDAC-4. In one embodiment thereof, the antisense oligonucleotide is a chimeric oligonucleotide. In another embodiment thereof, the antisense oligonucleotide is a hybrid oligonucleotide. In another embodiment thereof, the antisense oligonucleotide has a nucleotide sequence of from about 13 to about 35 nucleotides selected from the nucleotide sequence of SEQ ID NO:4. In still yet another embodiment thereof, the antisense oligonucleotide has a nucleotide sequence of from about 15 to about 26 nucleotides selected from the nucleotide sequence of SEQ ID NO:4. In another embodiment thereof, the antisense oligonucleotide has a nucleotide sequence of from about 20 to about 26 nucleotides selected from the nucleotide sequence of SEQ ID NO:4. In another embodiment thereof, the antisense oligonucleotide has a nucleotide sequence of from about 13 to about 35 nucleotides and which comprises the nucleotide sequence of SEQ ID NO:11. In still yet another embodiment thereof, the antisense oligonucleotide has a nucleotide sequence of from about 15 to about 26 nucleotides and which comprises the nucleotide sequence of SEQ ID NO:11. In another embodiment thereof, the antisense oligonucleotide has a nucleotide sequence of from about 20 to about 26 nucleotides and which comprises the nucleotide sequence of SEQ ID NO:11. In another embodiment thereof, the antisense oligonucleotide is SEQ ID NO:11. In another embodiment thereof, the antisense oligonucleotide has one or more phosphorothioate internucleoside linkages. In another embodiment thereof, the antisense oligonucleotide further comprises a length of 20-26 nucleotides. In still another embodiment thereof, the antisense oligonucleotide is modified such that the terminal four nucleotides at the 5′ end of the oligonucleotide and the terminal four nucleotides at the 3′ end of the oligonucleotide each have 2′-O— methyl groups attached to their sugar residues.

In certain preferred embodiments of the first aspect, the agent that inhibits the HDAC-4 isoform in a cell is a small molecule inhibitor that inhibits expression of a nucleic acid molecule encoding HDAC-4 isoform or activity of the HDAC-4 protein.

In a second aspect, the invention provides a method for inhibiting HDAC-4 activity in a cell, comprising contacting the cell with a specific inhibitor of HDAC-4, whereby HDAC-4 activity is inhibited. In an embodiment thereof, the invention provides method for inhibiting the HDAC-4 isoform in a cell, comprising contacting the cell with an antisense oligonucleotide complementary to a region of RNA that encodes a portion of HDAC-4 or to a region of double-stranded DNA that encodes a portion of HDAC-4, whereby HDAC-4 activity is inhibited. In one embodiment thereof, the cell is contacted with an HDAC-4 antisense oligonucleotide that is a chimeric oligonucleotide. In another embodiment thereof, the cell is contacted with an HDAC-4 antisense oligonucleotide that is a hybrid oligonucleotide. In another embodiment thereof, the antisense oligonucleotide has a nucleotide sequence of from about 13 to about 35 nucleotides selected from the nucleotide sequence of SEQ ID NO:4. In still yet another embodiment thereof, the antisense oligonucleotide has a nucleotide sequence of from about 15 to about 26 nucleotides selected from the nucleotide sequence of SEQ ID NO:4. In another embodiment thereof, the antisense oligonucleotide has a nucleotide sequence of from about 20 to about 26 nucleotides selected from the nucleotide sequence of SEQ ID NO:4. In yet another embodiment thereof, the cell is contacted with an HDAC-4 antisense oligonucleotide that has a nucleotide sequence length of from about 13 to about 35 nucleotides and which comprises the nucleotide sequence of SEQ ID NO:11. In another embodiment thereof, the cell is contacted with an HDAC-4 antisense oligonucleotide that has a nucleotide sequence length of from about 15 to about 26 nucleotides and which comprises the nucleotide sequence of SEQ ID NO:11. In another embodiment thereof, the cell is contacted with an HDAC-4 antisense oligonucleotide that is SEQ ID NO:11. In another embodiment thereof, the inhibition of HDAC-4 activity leads to the inhibition of cell proliferation in the contacted cell. In another embodiment thereof, the inhibition of HDAC-4 activity in the contacted cell further leads to growth retardation of the contacted cell. In another embodiment thereof, the inhibition of HDAC-4 activity in the contacted cell further leads to growth arrest of the contacted cell. In another embodiment thereof, the inhibition of HDAC-4 activity in the contacted cell further leads to programmed cell death of the contacted cell. In another embodiment thereof, the inhibition of HDAC-4 activity in the contacted cell further leads to necrotic cell death of the contacted cell. In certain embodiments thereof, the cell is a neoplastic cell which may be in an animal, including a human, and which may be in a neoplastic growth. In certain preferred embodiments, the method further comprises contacting the cell with an HDAC-4 small molecule inhibitor that interacts with and reduces the enzymatic activity of the HDAC-4 histone deacetylase isoform. In some embodiments thereof, the histone deacetylase small molecule inhibitor is operably associated with the antisense oligonucleotide.

In a third aspect, the invention provides a method for inhibiting neoplastic cell proliferation in an animal, comprising administering to an animal having at least one neoplastic cell present in its body a therapeutically effective amount of a specific inhibitor of HDAC-4, whereby neoplastic cell proliferation is inhibited in the animal. In an embodiment thereof, the invention provides a method for inhibiting neoplastic cell growth in an animal, comprising administering to an animal having at least one neoplastic cell present in its body a therapeutically effective amount of the antisense oligonucleotide of the first aspect of the invention with a pharmaceutically acceptable carrier for a therapeutically effective period of time. In an embodiment thereof, the animal is administered a chimeric HDAC-4 antisense oligonucleotide. In another embodiment thereof, the animal is administered a hybrid HDAC-4 antisense oligonucleotide. In another embodiment thereof, the antisense oligonucleotide has a nucleotide sequence of from about 13 to about 35 nucleotides selected from the nucleotide sequence of SEQ ID NO:4. In still yet another embodiment thereof, the antisense oligonucleotide has a nucleotide sequence of from about 15 to about 26 nucleotides selected from the nucleotide sequence of SEQ ID NO:4. In another embodiment thereof, the antisense oligonucleotide has a nucleotide sequence of from about 20 to about 26 nucleotides selected from the nucleotide sequence of SEQ ID NO:4. In another embodiment thereof, the animal is administered an HDAC-4 antisense oligonucleotide having a nucleotide sequence of from about 13 to about 35 nucleotides and which comprises the nucleotide sequence of SEQ ID NO:11. In another embodiment thereof, the animal is administered an HDAC-4 antisense oligonucleotide having a nucleotide sequence of from about 15 to about 26 nucleotides and which comprises the nucleotide sequence of SEQ ID NO:11. In another embodiment thereof, the animal is administered an HDAC-4 antisense oligonucleotide that is SEQ ID NO:11. In another embodiment thereof, the animal is a human. In another embodiment thereof, the method further comprises administering to an animal a therapeutically effective amount of an antisense oligonucleotide complementary to a region of RNA that encodes a portion of HDAC-1 or double-stranded DNA that encodes a portion of HDAC-1. In an embodiment thereof, the animal is administered a chimeric HDAC-1 antisense oligonucleotide. In another embodiment thereof, the animal is administered a hybrid HDAC-1 antisense oligonucleotide. In another embodiment thereof, the antisense oligonucleotide has a nucleotide sequence of from about 13 to about 35 nucleotides selected from the nucleotide sequence of SEQ ID NO:2. In still yet another embodiment thereof, the antisense oligonucleotide has a nucleotide sequence of from about 15 to about 26 nucleotides selected from the nucleotide sequence of SEQ ID NO:2. In another embodiment thereof, the antisense oligonucleotide has a nucleotide sequence of from about 20 to about 26 nucleotides selected from the nucleotide sequence of SEQ ID NO:2. In another embodiment thereof, the animal is administered an HDAC-1 antisense oligonucleotide having a nucleotide sequence of from about 13 to about 35 nucleotides and which comprises the nucleotide sequence of SEQ ID NO:5. In another embodiment thereof, the animal is administered an HDAC-1 antisense oligonucleotide having a nucleotide sequence of from about 15 to about 26 nucleotides and which comprises the nucleotide sequence of SEQ ID NO:5. In yet another embodiment thereof, the animal is administered an HDAC-1 antisense oligonucleotide that is SEQ ID NO:5.

In fourth aspect, the invention provides a method for inhibiting HDAC-4 activity in a cell, comprising contacting the cell with a small molecule inhibitor of HDAC-4, wherein HDAC-4 activity is inhibited.

In one embodiment thereof, the cell is contacted with a small molecule inhibitor having the structure

Cy-CH(OMe)—Y¹—C(O)—NH-Z (1)

wherein Cy is cycloalkyl, aryl, heteroaryl, or heterocyclyl, any of which may be optionally substituted; Y¹ is a C₄-C₆ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is selected from the group consisting of anilinyl, pyridyl, thiadiazolyl and —O-M, M being H or a pharmaceutically acceptable cation, wherein the anilinyl or pyridyl or thiadiazolyl may be optionally substituted.

In another embodiment thereof, the invention provides a method wherein the cell is contacted with a small molecule inhibitor having the structure

Cy-Y²—C(O)NH-Z (2)

wherein Cy is cycloalkyl, aryl, heteroaryl, or heterocyclyl, any of which may be optionally substituted; Y² is C₅-C₇ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is anilinyl or pyridyl, or thiadiazolyl, any of which may be optionally substituted.

In another embodiment thereof, the invention provides a method wherein the cell is contacted with a small molecule inhibitor having the structure

Cy-B—Y³—C(O)—NH-Z (3)

wherein Cy is cycloalkyl, aryl, heteroaryl, or heterocyclyl, any of which may be optionally substituted; B is selected from the group consisting of —CH(OMe), ketone and methylene; Y³ is a C₄-C₆ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is selected from the group consisting of anilinyl, pyridyl, thiadiazolyl and —O-M, M being H or a pharmaceutically acceptable cation, wherein the anilinyl or pyridyl or thiadiazolyl may be optionally substituted.

In another embodiment thereof, the invention provides a method wherein the cell is contacted with a small molecule inhibitor having the structure

Cy-L¹-Ar—Y¹—C(O)—NH-Z (4)

wherein Cy is cycloalkyl, aryl, heteroaryl, or heterocyclyl, any of which may be optionally substituted; L¹ is —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—, Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; Y¹ is a chemical bond or a straight- or branched-chain saturated alkylene, wherein said alkylene may be optionally substituted; and Z is selected from the group consisting of anilinyl, pyridyl, thiadiazolyl, and —O-M, M being H or a pharmaceutically acceptable cation; provided that when L¹ is —C(O)NH—, Y¹ is —(CH₂)ₙ—, n being 1, 2, or 3, and Z is —O-M, then Cy is not aminophenyl, dimethylaminophenyl, or hydroxyphenyl; and further provided that when L¹ is —C(O)NH— and Z is pyridyl, then Cy is not substituted indolinyl.

In another embodiment thereof, the invention provides a method wherein the cell is contacted with a small molecule inhibitor having the structure

Cy-L²Ar—Y²—C(O)NH-Z (5)

wherein Cy is cycloalkyl, aryl, heteroaryl, or heterocyclyl, any of which may be optionally substituted, provided that Cy is not a (spirocycloalkyl)heterocyclyl; L² is C₁-C₆ saturated alkylene or C₂-C₆ alkenylene, wherein the alkylene or alkenylene optionally may be substituted, provided that L² is not —C(O)—, and wherein one of the carbon atoms of the alkylene optionally may be replaced by a heteroatom moiety selected from the group consisting of O; NR′, R′ being alkyl, acyl, or hydrogen; S; S(O); or S(O)₂; Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and Y² is a chemical bond or a straight- or branched-chain saturated alkylene, which may be optionally substituted, provided that the alkylene is not substituted with a substituent of the formula —C(O)R wherein R comprises an α-amino acyl moiety; and Z is selected from the group consisting of anilinyl, pyridyl, thiadiazolyl, and —O-M, M being H or a pharmaceutically acceptable cation; provided that when the carbon atom to which Cy is attached is oxo substituted, then Cy and Z are not both pyridyl.

In another embodiment thereof, the invention provides a method wherein the cell is contacted with a small molecule inhibitor has the structure

Cy-L³-Ar—Y³—C(O)NH-Z (6)

wherein Cy is cycloalkyl, aryl, heteroaryl, or heterocyclyl, any of which may be optionally substituted, provided that Cy is not a (spirocycloalkyl)heterocyclyl; L³ is selected from the group consisting of (a) —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—; and (b) C₁-C₆ alkylene or C₂-C₆ alkenylene, wherein the alkylene or alkenylene optionally may be substituted, provided that L³ is not —C(O)—, and wherein one of the carbon atoms of the alkylene optionally may be replaced by O; NR′, R′ being alkyl, acyl, or hydrogen; S; S(O); or S(O)₂; Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and Y³ is C₂ alkenylene or C₂ alkynylene, wherein one or both carbon atoms of the alkenylene optionally may be substituted with alkyl, aryl, alkaryl, or aralkyl; and Z is selected from the group consisting of anilinyl, pyridyl, thiadiazolyl, and —O-M, M being H or a pharmaceutically acceptable cation; provided that when Cy is unsubstituted phenyl, Ar is not phenyl wherein L³ and Y³ are oriented ortho or meta to each other.

In another embodiment thereof, the invention provides a method wherein the cell is contacted with a small molecule inhibitor having the structure selected from the group consisting of

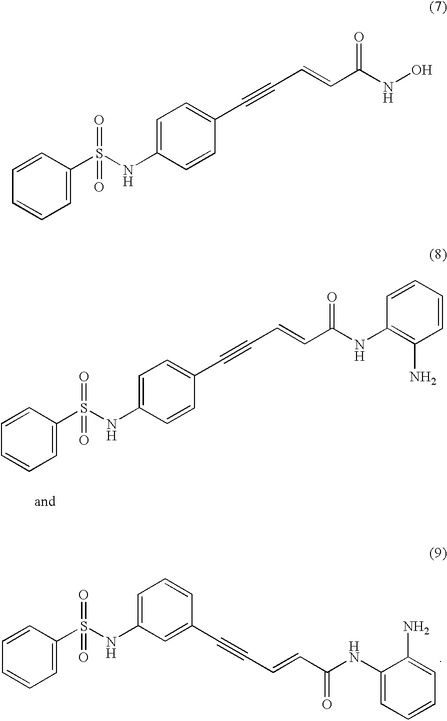

In another embodiment therein, the invention provides a method wherein the inhibition of HDAC-4 activity in the contacted cell further leads to an inhibition of cell proliferation in the contacted cell. In another embodiment therein, the invention provides a method wherein inhibition of HDAC-4 activity in the contacted cell further leads to growth retardation of the contacted cell. In another embodiment therein, the invention provides a method wherein inhibition of HDAC-4 activity in the contacted cell further leads to growth arrest of the contacted cell. In another embodiment therein, the invention provides a method wherein inhibition of HDAC-4 activity in the contacted cell further leads to programmed cell death of the contacted cell. In another embodiment therein, the invention provides a method wherein inhibition of HDAC-4 activity in the contacted cell further leads to necrotic cell death of the contacted cell. In another embodiment thereof, the contacted cell is a human cell.

In fifth aspect, the invention provides a method for inhibiting neoplastic cell proliferation in an animal, comprising administering to an animal having at least one neoplastic cell present in its body a therapeutically effective amount of a small molecule inhibitor of HDAC-4, whereby neoplastic cell proliferation is inhibited. In one embodiment thereof, the animal is administered a small molecule inhibitor having the structure

Cy-CH(OMe)—Y¹—C(O)—NH-Z (1)

wherein Cy is cycloalkyl, aryl, heteroaryl, or heterocyclyl, any of which may be optionally substituted; Y¹ is a C₄-C₆ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is selected from the group consisting of anilinyl, pyridyl, thiadiazolyl and —O-M, M being H or a pharmaceutically acceptable cation, wherein the anilinyl or pyridyl or thiadiazolyl may be optionally substituted. In another embodiment thereof, the invention provides a method wherein the animal is administered a small molecule inhibitor having the structure

Cy-Y²—C(O)NH-Z (2)

wherein Cy is cycloalkyl, aryl, heteroaryl, or heterocyclyl, any of which may be optionally substituted; Y² is C₅-C₇ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is anilinyl or pyridyl or thiadiazolyl, any of which may be optionally substituted. In another embodiment thereof, the invention provides a method wherein the animal is administered a small molecule inhibitor having the structure

Cy-B—Y³—C(O)—NH-Z (3)

wherein Cy is cycloalkyl, aryl, heteroaryl, or heterocyclyl, any of which may be optionally substituted; B is selected from the group consisting of —CH(OMe), ketone and methylene; Y³ is a C₄-C₆ alkylene, wherein said alkylene may be optionally substituted and wherein one of the carbon atoms of the alkylene optionally may be replaced by a heteroatom moiety selected from the group consisting of O; NR¹, R¹ being alkyl, acyl or hydrogen; S; S(O); or S(O)₂; and Z is selected from the group consisting of anilinyl, pyridyl, thiadiazolyl and —O-M, M being H or a pharmaceutically acceptable cation, wherein the anilinyl or pyridyl or thiadiazolyl may be optionally substituted. In another embodiment thereof, the invention provides a method wherein the animal is administered a small molecule inhibitor having the structure

Cy-L¹-Ar—Y¹—C(O)—NH-Z (4)

wherein Cy is cycloalkyl, aryl, heteroaryl, or heterocyclyl, any of which may be optionally substituted; L¹ is —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—; Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; Y¹ is a chemical bond or a straight- or branched-chain saturated alkylene, wherein said alkylene may be optionally substituted; and Z is selected from the group consisting of anilinyl, pyridyl, thiadiazolyl, and —O-M, M being H or a pharmaceutically acceptable cation; provided that when L¹ is —C(O)NH—, Y¹ is —(CH₂)ₙ—, n being 1, 2, or 3, and Z is —O-M, then Cy is not aminophenyl, dimethylaminophenyl, or hydroxyphenyl; and further provided that when L¹ is —C(O)NH— and Z is pyridyl, then Cy is not substituted indolinyl. In another embodiment thereof, the invention provides a method wherein the animal is administered a small molecule inhibitor having the structure

Cy-L²-Ar—Y²—C(O)NH-Z (5)

wherein Cy is cycloalkyl, aryl, heteroaryl, or heterocyclyl, any of which may be optionally substituted, provided that Cy is not a (spirocycloalkyl)heterocyclyl; L² is C₁-C₆ saturated alkylene or C₂-C₆ alkenylene, wherein the alkylene or alkenylene optionally may be substituted, provided that L² is not —C(O)—, and wherein one of the carbon atoms of the alkylene optionally may be replaced by a heteroatom moiety selected from the group consisting of O; NR′, R′ being alkyl, acyl, or hydrogen; S; S(O); or S(O)₂; Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and Y² is a chemical bond or a straight- or branched-chain saturated alkylene, which may be optionally substituted, provided that the alkylene is not substituted with a substituent of the formula —C(O)R wherein R comprises an α-amino acyl moiety; and Z is selected from the group consisting of anilinyl, pyridyl, thiadiazolyl, and —O-M, M being H or a pharmaceutically acceptable cation; provided that when the carbon atom to which Cy is attached is oxo substituted, then Cy and Z are not both pyridyl. In another embodiment thereof, the invention provides a method wherein the animal is administered a small molecule inhibitor having the structure

Cy-L³-Ar—Y³—C(O)NH-Z (6)

wherein Cy is cycloalkyl, aryl, heteroaryl, or heterocyclyl, any of which may be optionally substituted, provided that Cy is not a (spirocycloalkyl)heterocyclyl; L³ is selected from the group consisting of (a) —(CH₂)ₘ—W—, where m is 0, 1, 2, 3, or 4, and W is selected from the group consisting of —C(O)NH—, —S(O)₂NH—, —NHC(O)—, —NHS(O)₂—, and —NH—C(O)—NH—; and (b) C₁-C₆ alkylene or C₂-C₆ alkenylene, wherein the alkylene or alkenylene optionally may be substituted, provided that L³ is not —C(O)—, and wherein one of the carbon atoms of the alkylene optionally may be replaced by O; NR′, R′ being alkyl, acyl, or hydrogen; S; S(O); or S(O)₂; Ar is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and Y³ is C₂ alkenylene or C₂ alkynylene, wherein one or both carbon atoms of the alkenylene optionally may be substituted with alkyl, aryl, alkaryl, or aralkyl; and Z is selected from the group consisting of anilinyl, pyridyl, thiadiazolyl, and —O-M, M being H or a pharmaceutically acceptable cation; provided that when Cy is unsubstituted phenyl, Ar is not phenyl wherein L³ and Y³ are oriented ortho or meta to each other. In another embodiment thereof, the invention provides a method wherein the animal is administered a small molecule inhibitor having the structure selected from the group consisting of

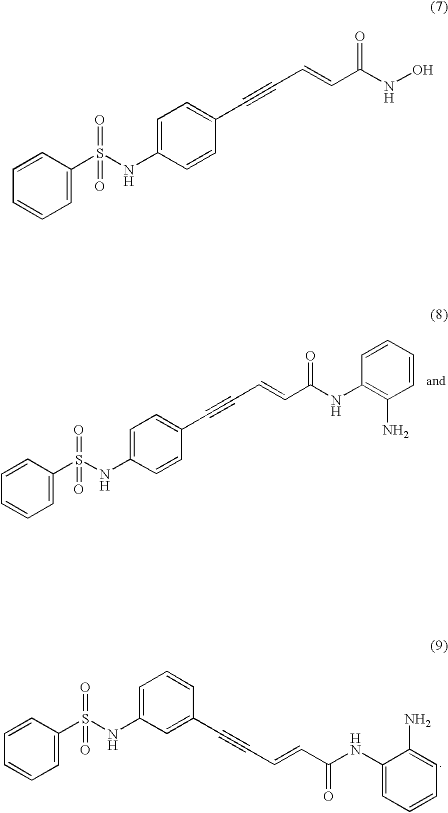

In another embodiment thereof, the invention provides a method wherein the animal administered a small molecule inhibitor is a human.

In a sixth aspect, the invention provides a method for inhibiting the induction of cell proliferation, comprising contacting a cell with an antisense oligonucleotide that inhibits the expression of HDAC-4 and/or contacting a cell with a small molecule inhibitor of HDAC-4. In certain preferred embodiments, the cell is a neoplastic cell, and the induction of cell proliferation is tumorigenesis.

In a seventh aspect, the invention provides a method for identifying a small molecule histone deacetylase inhibitor that inhibits the HDAC-4 isoform, the isoform being required for the induction of cell proliferation. The method comprises contacting the HDAC-4 isoform with a candidate small molecule inhibitor and measuring the enzymatic activity of the contacted histone deacetylase isoform, wherein a reduction in the enzymatic activity of the contacted HDAC-4 isoform identifies the candidate small molecule inhibitor as a small molecule histone deacetylase inhibitor of the HDAC-4 isoform.

In an eighth aspect, the invention provides a method for identifying a small molecule histone deacetylase inhibitor that inhibits HDAC-4 isoform, which is involved in the induction of cell proliferation. The method comprises contacting a cell with a candidate small molecule inhibitor and measuring the enzymatic activity of the contacted histone deacetylase isoform, wherein a reduction in the enzymatic activity of the HDAC-4 isoform identifies the candidate small molecule inhibitor as a small molecule histone deacetylase inhibitor of HDAC-4.

In a ninth aspect, the invention provides a small molecule histone deacetylase inhibitor identified by the method of the seventh or the eighth aspect of the invention. Preferably, the histone deacetylase small molecule inhibitor is substantially pure.

In a tenth aspect, the invention provides a method for inhibiting cell proliferation in a cell comprising, contacting a cell with at least two reagents selected from the group consisting of an antisense oligonucleotide that inhibits expression of HDAC-4 isoform, a small molecule histone deacetylase inhibitor that inhibits expression or activity of HDAC-4 isoform, an antisense oligonucleotide that inhibits expression of the HDAC-1 isoform, a small molecule histone deacetylase inhibitor that inhibits the expression or the activity of the HDAC-1 isoform, an antisense oligonucleotide that inhibits expression of a DNA methyltransferase, and a small molecule DNA methyltransferase inhibitor. In certain embodiments, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional embodiments, the reagents selected from the group are operably associated.

In an eleventh aspect, the invention provides a method of inhibiting neoplastic cell growth, comprising contacting a cell with at least two reagents selected from the group consisting of an antisense oligonucleotide that inhibits expression of HDAC-4 isoform, a small molecule histone deacetylase inhibitor that inhibits the expression or the activity of HDAC-4 isoform, an antisense oligonucleotide that inhibits expression of the HDAC-1 isoform, a small molecule histone deacetylase inhibitor that inhibits expression or activity of the HDAC-1 isoform, an antisense oligonucleotide that inhibits expression of a DNA methyltransferase, and a small molecule DNA methyltransferase inhibitor. In some embodiments, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

### BRIEF DESCRIPTION OF THE DRAWINGS

FIG. 1 AS1 and AS2 can inbibit HDAC4 expression at RNA level in a dose-dependent manner. Human cancer A549 cells were treated with escalating doses of AS1, AS2 or MM2 oligos for 24 hours. Total RNAs were harvested for Northern analysis.

FIG. 2 AS1 and AS2 can inbibit HDAC4 expression at protein level. Human cancer A549 cells were treated with AS1, AS2 or MM2 oligos for 48 hours. Whole cell lysates were analyzed by Western blotting using antibodies specific against human HDAC4.

FIG. 3 Growth curve of human cancer cells A549 treated with HDAC4 AS1 or AS2. Cells were plated at 2.5×10⁵/10 cm dish at 0 hour time point. Cells were treated with 50 nM oligos at 24 and 48 hours. Cells were counted at 24, 48 and 72 hours by trypan blue exclusion.

FIG. 4 Growth curve of human cancer cells Du145 treated with HDAC4 AS1 or AS2. Cells were plated at 2.5×10⁵/10 cm dish at day 0. Cells were treated with 50 nM oligos at day 1, day 2 and day 3. Cells were counted at day 1, day 2, day 3 and day 4 by trypan blue exclusion.

FIG. 5 Graphic representation demonstrating the apoptotic effect of HDAC isotype-specific antisense oligos on human A549 cancer cells.

FIG. 6 is a a graphic representation demonstrating the cell cycle blocking effect of HDAC-4 antisense oligos on human A549 cancer cells.

FIG. 7 is a representation of an RNAse protection assay demonstrating the effect of HDAC isotype-specific antisense oligos on HDAC isotype mRNA expression in human A549 cells.

FIG. 8 is a representation of a Western blot demonstrating that treatment of human A549 cells with HDAC-4 antisense oligos induces the expression of the p21 protein.

### DETAILED DESCRIPTION OF THE PREFERRED EMBODIMENTS

The patent and scientific literature referred to herein establishes knowledge that is available to those with skill in the art. The issued patents, applications, and references, including GenBank database sequences, that are cited herein are hereby incorporated by reference to the same extent as if each was specifically and individually indicated to be incorporated by reference.

The invention provides methods and reagents for modulating histone deacetylase (HDAC) isoforms, particularly HDAC-1 and HDAC-4, by inhibiting expression at the nucleic acid level or by inhibiting enzymatic activity at the protein level. The invention provides for the specific inhibition of specific histone deacetylase isoforms involved in tumorigenesis, and thus provides a treatment for cancer. The invention further provides for the specific inhibition of specific HDAC isoforms involved in cell proliferation and thus provides a treatment for cell proliferative disorders.

The inventors have made the surprising discovery that the specific inhibition of HDAC-4 dramatically induces apoptosis and growth arrest in cancerous cells. This discovery has been exploited to develop the present invention which, in a first aspect, provides agents that inhibit the HDAC-4 isoform.

In certain preferred embodiments of the first aspect of the invention, the agent that inhibits the HDAC-4 isoform is an oligonucleotide that inhibits expression of a nucleic acid molecule encoding HDAC-4 isoform. The HDAC-4 nucleic acid molecule may be genomic DNA (e.g., a gene), cDNA, or RNA. In some embodiments, the oligonucleotide inhibits transcription of mRNA encoding the HDAC-4 isoform. In other embodiments, the oligonucleotide inhibits translation of the HDAC-4 isoform. In certain embodiments the oligonucleotide causes the degradation of the nucleic acid molecule. Preferred antisense oligonucleotides have potent and specific antisense activity at nanomolar concentrations.

In certain preferred embodiments, the agent that inhibits the HDAC-4 isoform is a small molecule inhibitor that inhibits expression of a nucleic acid molecule encoding HDAC-4 isoform or activity of the HDAC-4 protein.

The term “small molecule” as used in reference to the inhibition of histone deacetylase is used to identify a compound having a molecular weight preferably less than 1000 Da, more preferably less than 800 Da, and most preferably less than 600 Da, which is capable of interacting with a histone deacetylase and inhibiting the expression of a nucleic acid molecule encoding an HDAC isoform or activity of an HDAC protein. Inhibiting histone deacetylase enzymatic activity means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. In a particularly preferred embodiment, the small molecule inhibitor of HDAC is an inhibitor of HDAC-1 and/or HDAC-4. Most prefered are small molecule inhibitors of HDAC-4.

Preferably, such inhibition is specific, i.e., the histone deacetylase inhibitor reduces the ability of a histone deacetylase to remove an acetyl group from a histone at a concentration that is lower than the concentration of the inhibitor that is required to produce another, unrelated biological effect. Preferably, the concentration of the inhibitor required for histone deacetylase inhibitory activity is at least 2-fold lower, more preferably at least 5-fold lower, even more preferably at least 10-fold lower, and most preferably at least 20-fold lower than the concentration required to produce an unrelated biological effect.

Preferred agents that inhibit HDAC-4 inhibit growth of human cancer cells, independent of their p53 status. These agents induce apoptosis in cancer cells and cause growth arrest. They also can induce transcription of p21^{WAF1} (a tumor suppressor gene), Bax, an extremely important gene involved in apoptosis regulation and GADD45, a stress-induced gene and important regulator of cell growth. These agents may exhibit both in vitro and in vivo anti-tumor activity. Inhibitory agents that achieve one or more of these results are considered within the scope of this aspect of the invention.

The antisense oligonucleotides according to the invention are complementary to a region of RNA or to a region of double-stranded DNA that encodes a portion of one or more histone deacetylase isoforms (taking into account that homology between different isoforms may allow a single antisense oligonucleotide to be complementary to a portion of more than one isoform). For purposes of the invention, the term “oligonucleotide” includes polymers of two or more deoxyribonucleosides, ribonucleosides, or any combination thereof. Preferably, such oligonucleotides have from about 6 to about 50 nucleoside residues, and most preferably from about 12 to about 30 nucleoside residues. The nucleoside residues may be coupled to each other by any of the numerous known internucleoside linkages. Such internucleoside linkages include without limitation phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleotide linkages. These internucleoside linkages preferably are phosphotriester, phosphorothioate, or phosphoramidate linkages, or combinations thereof.

Preferably, the oligonucleotides may also contain 2′-O-substituted ribonucleotides. For purposes of the invention the term “2″-O-substituted” means substitution of the 2′ position of the pentose moiety with an —O-lower alkyl group containing 1-6 saturated or unsaturated carbon atoms, or with an —O-aryl or allyl group having 2-6 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups; or such 2′ substitution may be with a hydroxy group (to produce a ribonucleoside), an amino or a halo group, but not with a 2′-H group. The term “alkyl” as employed herein refers to straight and branched chain aliphatic groups having from 1 to 12 carbon atoms, preferably 1-8 carbon atoms, and more preferably 1-6 carbon atoms, which may be optionally substituted with one, two or three substituents. Unless otherwise apparent from context, the term “alkyl” is meant to include saturated, unsaturated, and partially unsaturated aliphatic groups. When unsaturated groups are particularly intended, the terms “alkenyl” or “alkynyl” will be used. When only saturated groups are intended, the term “saturated alkyl” will be used. Preferred saturated alkyl groups include, without limitation, methyl, ethyl, propyl, isopropyl, butyl, isobutyl, sec-butyl, tert-butyl, pentyl, and hexyl.

The term oligonucleotide also encompasses such polymers having chemically modified bases or sugars and/or having additional substituents including, without limitation, lipophilic groups, intercalating agents, diamines, and adamantane. The term oligonucleotide also encompasses such polymers as PNA and LNA.

For purposes of the invention, the term “complementary” means having the ability to hybridize to a genomic region, a gene, or an RNA transcript thereof, under physiological conditions. Such hybridization is ordinarily the result of base-specific hydrogen bonding between complementary strands, preferably to form Watson-Crick or Hoogsteen base pairs, although other modes of hydrogen bonding, as well as base stacking can lead to hybridization. As a practical matter, such hybridization can be inferred from the observation of specific gene expression inhibition, which may be at the level of transcription or translation (or both).

Particularly preferred antisense oligonucleotides utilized in this aspect of the invention include chimeric oligonucleotides and hybrid oligonucleotides.

For purposes of the invention, a “chimeric oligonucleotide” refers to an oligonucleotide having more than one type of internucleoside linkage. One preferred embodiment of such a chimeric oligonucleotide is a chimeric oligonucleotide comprising internucleoside linkages, phosphorothioate, phosphorodithioate, internucleoside linkages and phosphodiester, preferably comprising from about 2 to about 12 nucleotides. Some useful oligonucleotides of the invention have an alkylphosphonate-linked region and an alkylphosphonothioate region (see e.g., Pederson et al. U.S. Pat. Nos. 5,635,377 and 5,366,878). Preferably, such chimeric oligonucleotides contain at least three consecutive internucleoside linkages that are phosphodiester and phosphorothioate linkages, or combinations thereof.

For purposes of the invention, a “hybrid oligonucleotide” refers to an oligonucleotide having more than one type of nucleoside. One preferred embodiment of such a hybrid oligonucleotide comprises a ribonucleotide or 2′-O-substituted ribonucleotide region, preferably comprising from about 2 to about 12 2′-O-substituted nucleotides, and a deoxyribonucleotide region. Preferably, such a hybrid oligonucleotide contains at least three consecutive deoxyribonucleosides and contains ribonucleosides, 2′-O-substituted ribonucleosides, or combinations thereof (see e.g., Metelev and Agrawal, U.S. Pat. Nos. 5,652,355 and 5,652,356).

The exact nucleotide sequence and chemical structure of an antisense oligonucleotide utilized in the invention can be varied, so long as the oligonucleotide retains its ability to modulate expression of the target sequence, e.g., the HDAC-4 or the HDAC-1 isoform. This is readily determined by testing whether the particular antisense oligonucleotide is active by quantitating the amount of mRNA encoding the HDAC-4 or the HDAC-1 isoform, quantitating the amount of the HDAC-4 or the HDAC-1 isoform protein, quantitating the the HDAC-4 or the HDAC-1 isoform enzymatic activity, or quantitating the ability of the the HDAC-4 or the HDAC-1 isoform, for example, to inhibit cell growth in a an in vitro or in vivo cell growth assay, all of which are described in detail in this specification. The term “inhibit expression” and similar terms used herein are intended to encompass any one or more of these parameters.

Antisense oligonucleotides according to the invention may conveniently be synthesized on a suitable solid support using well-known chemical approaches, including H-phosphonate chemistry, phosphoramidite chemistry, or a combination of H-phosphonate chemistry and phosphoramidite chemistry (i.e., H-phosphonate chemistry for some cycles and phosphoramidite chemistry for other cycles). Suitable solid supports include any of the standard solid supports used for solid phase oligonucleotide synthesis, such as controlled-pore glass (CPG) (see, e.g., Pon, R. T., Meth. Molec. Biol. 20:465-496, 1993).

Antisense oligonucleotides according to the invention are useful for a variety of purposes. For example, they can be used as “probes” of the physiological function of specific histone deacetylase isoforms by being used to inhibit the activity of specific histone deacetylase isoforms in an experimental cell culture or animal system and to evaluate the effect of inhibiting such specific histone deacetylase isoform activity. This is accomplished by administering to a cell or an animal an antisense oligonucleotide that inhibits one or more histone deacetylase isoform expression according to the invention and observing any phenotypic effects. In this use, the antisense oligonucleotides used according to the invention are preferable to traditional “gene knockout” approaches because they are easier to use, and because they can be used to inhibit specific histone deacetylase isoform activity at selected stages of development or differentiation.

Preferred antisense oligonucleotides of the invention inhibit either the transcription of a nucleic acid molecule encoding the the HDAC-4 or the HDAC-1 isoform, and/or the translation of a nucleic acid molecule encoding the the HDAC-4 or the HDAC-1, and/or lead to the degradation of such nucleic acid molecules. HDAC-4- or HDAC-1-encoding nucleic acid molecules may be RNA or double stranded DNA regions and include, without limitation, intronic sequences, untranslated 5′ and 3′ regions, intron-exon boundaries, as well as coding sequences from the HDAC-4 or the HDAC-1 isoform genes. For human sequences, see e.g., Yang et al., Proc. Natl. Acad. Sci. USA 93(23):2845-12850, 1996; Furukawa et al., Cytogenet. Cell Genet. 73(1-2):130-133, 1996; Yang et al., J. Biol. Chem. 272(44):28001-28007, 1997; Betz et al., Genomics 52(2):245-246, 1998; Taunton et al., Science 272(5260):408411, 1996; and Dangond et al., Biochem. Biophys. Res. Commun. 242(3):648-652, 1998).

Antisense oligonucleotides for human HDAC isotype polynucleotides may be designed from known HDAC isotype sequence data. For example, the following amino acid sequences are available from GenBank for HDAC-1, HDAC-2, HDAC-3, HDAC-4, HDAC-5, HDAC-6, HDAC-7, and HDAC-8: AAC50475, AAC50814, AAC98927, BAA22957, AB011172, AAD29048, AAF63491, and AAF73076, respectively, and the following nucleotide sequences are available from GenBank for HDAC-1, HDAC-2, HDAC-3, HDAC-4, HDAC-5, HDAC-6, HDAC-7, and HDAC-8: U50079, U31814, AF039703, AB006626, AF039691, AJ011972, AF239243, and AF230097, respectively.

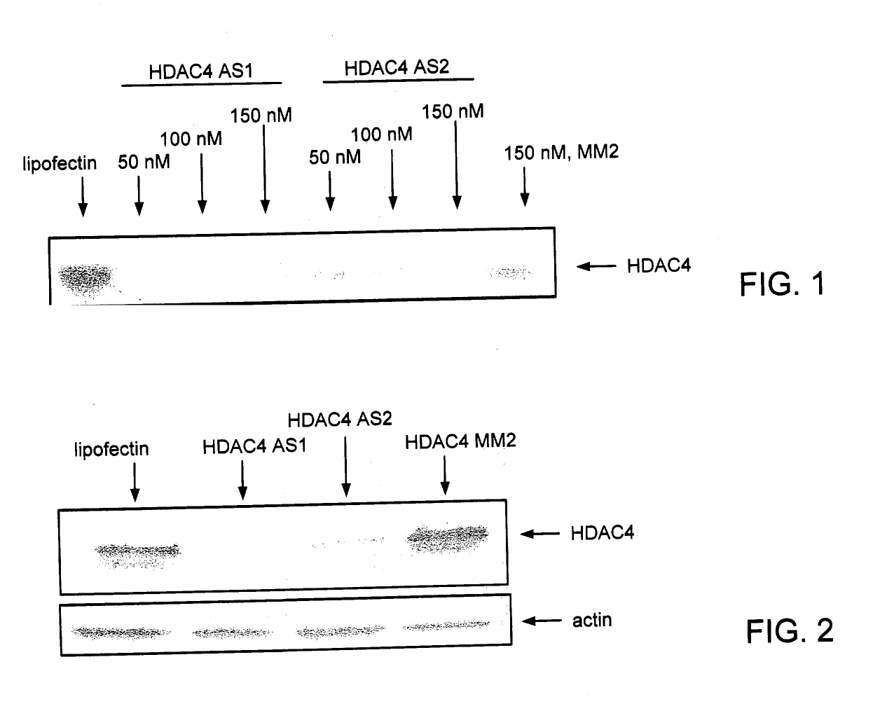

Particularly preferred non-limiting examples of antisense oligonucleotides of the invention are complementary to a region of RNA or to a region of double-stranded DNA encoding the HDAC-4 or the HDAC-1 isoform, (see e.g., GenBank Accession No. U50079 for human HDAC-1 (FIG. 1B), and GenBank Accession No. AB006626 for human HDAC-4 (FIG. 2B)).

The sequences encoding histone deacetylases from many non-human animal species are also known (see, for example, GenBank Accession Nos. X98207 (murine HDAC-1) and AF006602 (murine HDAC-4)). Accordingly, the antisense oligonucleotides of the invention may also be complementary to a region of RNA or to a region of double-stranded DNA that encode the HDAC-4 or the HDAC-1 isoform from non-human animals. Antisense oligonucleotides according to these embodiments are useful as tools in animal models for studying the role of specific histone deacetylase isoforms.

Particularly, preferred oligonucleotides have nucleotide sequences of from about 13 to about 35 nucleotides which include the nucleotide sequences shown in Table I below.

These oligonucleotides have nucleotide sequences of from about 15 to about 26 nucleotides of the nucleotide sequences shown below in Table I. Most preferably, the oligonucleotides shown below have phosphorothioate backbones, are 20-26 nucleotides in length, and are modified such that the terminal four nucleotides at the 5′ end of the oligonucleotide and the terminal four nucleotides at the 3′ end of the oligonucleotide each have 2′-O— methyl groups attached to their sugar residues.

TABLE 1
HDAC isotype-specific antisense and mismatch oligos

| | | Accession | Nucleotide | | position within |
|---|---|---|---|---|---|
| Oligo | Target | Number | Position | Sequence | Gene |
| HDAC1 AS1 | Human | U50079 | 1585-1604 | 5′-GAAACGTGAGGGACTCAGCA-3′ | 3′-UTR |
| | HDAC1 | | | | |
| HDAC1 AS2 | Human | U50079 | 1565-1584 | 5′-GGAAGCCAGAGCTGGAGAGG-3′ | 3′-UTR |
| | HDAC1 | | | | |
| HDAC1 MM | Human | U50079 | 1585-1604 | 5′-GTTAGGTGAGGCACTGAGGA-3′ | 3′-UTR |
| | HDAC1 | | | | |
| HDAC2 AS | Human | U31814 | 1643-1622 | 5′-GCTGAGCTGTTCTGATTTGG-3′ | 3′-UTR |
| | HDAC2 | | | | |
| HDAC2 MM | Human | U31814 | 1643-1622 | 5′-CGTGAGCACTTCTCATTTCC-3′ | 3′-UTR |
| | HDAC2 | | | | |
| HDAC3 AS | Human | AF039703 | 1276-1295 | 5′-CGCTTTCCTTGTCATTGACA-3′ | 3′-UTR |
| | HDAC3 | | | | |
| HDAC3 MM | Human | AF039703 | 1276-1295 | 5′-GCCTTTCCTACTCATTGTGT-3′ | 3′-UTR |
| | HDAC3 | | | | |
| HDAC4 AS1 | Human | AB006626 | 514-33 | 5′-GCTGCCTGCCGTGCCCACCC-3′ | 5′-UTR |
| | HDAC4 | | | | |
| HDAC4 | Human | AB006626 | 514-33 | 5′-CGTGCCTGCGCTGCCCACGG-3′ | 5′-UTR |
| MM1 | HDAC4 | | | | |
| HDAC4 AS2 | Human | AB006626 | 7710-29 | 5′-TACAGTCCATGCAACCTCCA-3′ | 3′-UTR |
| | HDAC4 | | | | |
| HDAC4 | Human | AB006626 | 7710-29 | 5′-ATCAGTCCAACCAACCTCGT-3′ | 3′-UTR |
| MM2 | HDAC4 | | | | |
| HDAC5 AS1 | Human | BE794912 | 1-20 | 5′-GCAGCGGCGGCAGCACCTCC-3′ | 5′-UTR |
| | HDAC5 | | | | |
| HDAC5 AS2 | Human | AF039691 | 2663-2682 | 5′-CTTCGGTCTCACCTGCTTGG-3′ | 3′-UTR |
| | HDAC5 | | | | |
| HDAC6 AS | Human | AJ011972 | 3791-3810 | 5′-CAGGCTGGAATGAGCTACAG-3′ | 3′-UTR |
| | HDAC6 | | | | |
| HDAC6 MM | Human | AJ011972 | 3791-3810 | 5′-GACGCTGCAATCAGGTAGAC-3′ | 3′-UTR |
| | HDAC6 | | | | |
| HDAC7 AS1 | Human | AF239243 | 65-84 | 5′-CAGGCTCACTTGACAATGGC-3′ | 5′-UTR |
| | HDAC7 | | | | |
| HDAC7 AS2 | Human | AF239243 | 2896-2915 | 5′-CTTCAGCCAGGATGCCCACA-3′ | 5′-UTR |
| | HDAC7 | | | | |
| HDAC8 AS1 | Human | AF230097 | 51-70 | 5′-CTCCGGCTCCTCCATCTTCC-3′ | 5′-UTR |
| | HDAC8 | | | | |
| HDAC8 AS2 | Human | AF230097 | 1328-1347 | 5′-AGCCAGCTGCCACTTGATGC-3′ | 3′-UTR |
| | HDAC8 | | | | |

The antisense oligonucleotides according to the invention may optionally be formulated with any of the well known pharmaceutically acceptable carriers or diluents (see preparation of pharmaceutically acceptable formulations in, e.g., Remington's Pharmaceutical Sciences, 18th Edition, ed. A. Gennaro, Mack Publishing Co., Easton, Pa., 1990), with the proviso that such carriers or diluents not affect their ability to modulate HDAC activity.

In certain preferred embodiments, the agent that inhibits the HDAC-4 and/or HDAC-1 isoform is a small molecule. In certain preferred embodiments, the small molecule inhibits the enzymatic activity of the HDAC-4 or HDAC-1 isoform.

Certain preferred small molecule inhibitors of the HDAC-4 and/or HDAC-1 isoform include compounds having the formula (1):

Cy-CH(OMe)—Y¹—C(O)—NH-Z (1)

wherein:

Cy is cycloalkyl, aryl, heteroaryl, or heterocyclyl, any of which may optionally be substituted;

Y¹ is a C₄-C₆ alkylene which optionally may be substituted and wherein one of the carbon atoms of the alkylene optionally may be replaced by a heteroatom moiety such as O, NR¹ (R¹ being alkyl, acyl or hydrogen) S, S(O), or S(O)₂; and

Z is selected from the group consisting of anilinyl, pyridyl, thiadiazolyl and —O-M, M being H or a pharmaceutically acceptable cation, wherein the anilinyl or pyridyl or thiadiazolyl may be optionally substituted.

An “alkylene” group is an alkyl group, as defined hereinabove, that is positioned between and serves to connect two other chemical groups. Preferred alkylene groups include, without limitation, methylene, ethylene, propylene, and butylene.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

An “aryl” group is a C₆-C₁₄ aromatic moiety comprising one to three aromatic rings, which may be optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl. An “alkaryl” or “alkylaryl” group is an aryl group having one or more alkyl substituents. Examples of alkaryl groups include, without limitation, tolyl, xylyl, mesityl, ethylphenyl, tert-butylphenyl, and methylnaphthyl.

An “arylene” group is an aryl group, as defined hereinabove, that is positioned between and serves to connect two other chemical groups. Preferred arylene groups include, without limitation, phenylene and naphthylene. The term “arylene” is also meant to include heteroaryl bridging groups, including, but not limited to, benzothienyl, benzofuryl, quinolyl, isoquinolyl, and indolyl.

A “heterocyclyl” or “heterocyclic” group is a ring structure having from about 3 to about 8 atoms, wherein one or more atoms are selected from the group consisting of N, O, and S. The heterocyclic group may be optionally substituted on carbon at one or more positions. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl or heteroaryl group. Examples of such fused heterocyles include, without limitation, tetrahydroquinoline and dihydrobenzofuran.

As used herein, the term “heteroaryl” refers to groups having 5 to 14 ring atoms, preferably 5, 6, 9, or 10 ring atoms; having 6, 10, or 14 π electrons shared in a cyclic array; and having, in addition to carbon atoms, between one and about three heteroatoms selected from the group consisting of N, O, and S. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

As employed herein, a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

The term “halogen” or “halo” as employed herein refers to chlorine, bromine, fluorine, or iodine.

As herein employed, the term “acyl” refers to an alkylcarbonyl or arylcarbonyl substituent.

The term “acylamino” refers to an amide group attached at the nitrogen atom. The term “carbamoyl” refers to an amide group attached at the carbonyl carbon atom. The nitrogen atom of an acylamino or carbamoyl substituent may be additionally substituted. The term “sulfonamido” refers to a sulfonamide substituent attached by either the sulfur or the nitrogen atom. The term “amino” is meant to include NH₂, alkylamino, arylamino, and cyclic amino groups.

The term “ureido” as employed herein refers to a substituted or unsubstituted urea moiety.

In another embodiment, the small molecule inhibitors of the HDAC-4 and/or HDAC-1 isoform are represented by formula (2):

Cy-Y²—C(O)NH-Z (2)

wherein:

Cy is cycloalkyl, aryl, heteroaryl, or heterocyclyl, any of which may optionally be substituted;

Y² is C₅-C₇ alkylene which may be optionally substituted and wherein one of the carbon atoms of the alkylene optionally may be replaced by a heteroatom moiety such as O, NR¹ (R¹ being alkyl, acyl or hydrogen), S, S(O), or S(O)₂; and

Z is anilinyl or pyridyl or thiadiazolyl, any of which may optionally be optionally substituted. In another embodiment, preferred small molecule inhibitors of the HDAC-4 and/or HDAC-1 isoform include compounds having the formula (3):

Cy-B—Y³—C(O)—NH-Z (3)

wherein:

Cy is cycloalkyl, aryl, heteroaryl, or heterocyclyl, any of which may optionally be substituted;

B is —CH(OMe), ketone, or methylene;

Y³ is a C₄-C₆ alkylene which may be optionally substituted, and wherein one of the carbon atoms of the alkylene optionally may be replaced by a heteroatom moiety such as O, NR¹ (R¹ being alkyl, acyl or hydrogen), S, S(O), or S(O)₂; and

Z is anilinyl, pyridyl, thiadiazolyl or —O-M (M being H or a pharmaceutically acceptable cation), wherein the anilinyl or pyridyl or thiadiazolyl optionally may be substituted.

In another embodiment, the inhibitors of the HDAC-4 and/or HDAC-1 isoform are represented by formula (4):

Cy-L¹-Ar—Y¹—C(O)—NH-Z (4)

wherein:

Cy is cycloalkyl, aryl, heteroaryl, or heterocyclyl, any of which optionally may be substituted;

L¹ is —(CH₂)ₘ—W—, where m is 0, 1, 2, 3, or 4, and W is —C(O)NH—, —S(O)₂NH—, —NHC(O)—, —NHS(O)₂—, or —NH—C(O)—NH—;

Ar is arylene which may be additionally substituted and optionally may be fused to an aryl or heteroaryl ring, or to a saturated or partially unsaturated cycloalkyl or heterocyclic ring, any of which optionally may be substituted;

Y¹ is a chemical bond or a straight- or branched-chain saturated alkylene, which optionally may be substituted; and

Z is anilinyl, pyridyl, thiadiazolyl, or —O-M (M being H or a pharmaceutically acceptable cation);

provided that when L¹ is —C(O)NH—, Y¹ is —(CH₂)ₙ— (n being 1, 2, or 3), and Z is —O-M, then Cy is not aminophenyl, dimethylaminophenyl, or hydroxyphenyl; and further provided that when L¹ is —C(O)NH— and Z is pyridyl, then Cy is not substituted indolinyl.

In another embodiment, the inhibitors of the HDAC-4 and/or HDAC-1 isoform are represented by formula (5):

Cy-L²-Ar—Y²—C(O)NH-Z (5)

wherein:

Cy is cycloalkyl, aryl, heteroaryl, or heterocyclyl, any of which optionally may be substituted, provided that Cy is not a (spirocycloalkyl)heterocyclyl;

L² is C₁-C₆ saturated alkylene or C₂-C₆ alkenylene, wherein the alkylene or alkenylene optionally may be substituted, provided that L² is not —C(O)—, and wherein one of the carbon atoms of the alkylene optionally may be replaced by a heteroatom moiety such as O, NR′ (R′ being alkyl, acyl, or hydrogen), S, S(O), or S(O)₂;

Ar is arylene which optionally may be additionally substituted and optionally may be fused to an aryl or heteroaryl ring, or to a saturated or partially unsaturated cycloalkyl or heterocyclic ring, any of which optionally may be substituted; and

Y² is a chemical bond or a straight- or branched-chain saturated alkylene which may be optionally substituted, provided that the alkylene is not substituted with a substituent of the formula —C(O)R, wherein R comprises an α-amino acyl moiety; and

Z is anilinyl, pyridyl, thiadiazolyl, or —O-M (M being H or a pharmaceutically acceptable cation);

provided that when the carbon atom to which Cy is attached is oxo-substituted, then Cy and Z are not both pyridyl.

In another embodiment, the inhibitors of the HDAC-4 and/or HDAC-1 isoform are represented by formula (6):

Cy-L³-Ar—Y³—C(O)NH-Z (6)

wherein:

Cy is cycloalkyl, aryl, heteroaryl, or heterocyclyl, any of which optionally may be substituted, provided that Cy is not a (spirocycloalkyl)heterocyclyl;

L³ is:

(a) —(CH₂)ₘ—W—, where m is 0, 1, 2, 3, or 4, and W is —C(O)NH—, S(O)₂NH—, —NHC(O)—, —NHS(O)₂—, or —NH—C(O)—NH—, or

(b) C₁-C₆ alkylene or C₂-C₆ alkenylene, wherein the alkylene or alkenylene optionally may be substituted, provided that L³ is not —C(O)—, and wherein one of the carbon atoms of the alkylene optionally may be replaced by O; NR′, R′ being alkyl, acyl, or hydrogen; S; S(O); or S(O)₂;

Ar is arylene which optionally may be additionally substituted and optionally may be fused to an aryl or heteroaryl ring, or to a saturated or partially unsaturated cycloalkyl or heterocyclic ring, any of which optionally may be substituted; and

Y³ is C₂ alkenylene or C₂ alkynylene, wherein one or both carbon atoms of the alkenylene optionally may be substituted with alkyl, aryl, alkaryl, or aralkyl; and

Z is anilinyl, pyridyl, thiadiazolyl, or —O-M (M being H or a pharmaceutically acceptable cation);

provided that when Cy is unsubstituted phenyl, Ar is not phenyl wherein L³ and Y³ are oriented ortho or meta to each other.

In another embodiment, the small molecule inhibitors of the HDAC-4 and/or HDAC-1 isoform have the structure selected from the group consisting of

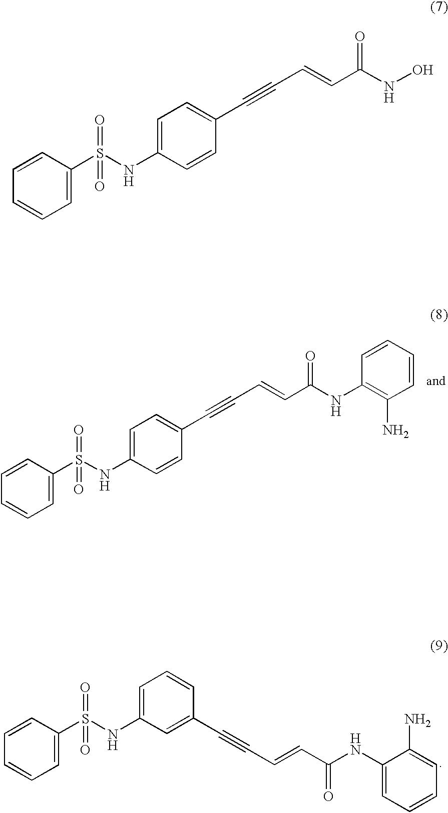

Non-limiting examples of small molecule inhibitors for use in the methods of the invention are presented in Table 2.

TABLE 2
Properties of Selected MG Anilides in
vitro and in vivo (shown in μM)

| | | Enzyme IC50 (μM) | | | | | | | cell | p21 | % inh. of tumor | | |
|---|---|---|---|---|---|---|---|---|---|---|---|---|---|
| | | HD- | HD- | HD- | HD- | HD- | | | cycle | in- | formation in vivo | | |
| MG | | AC | AC | AC | AC | AC | H4- | M- | arrest | duc- | | | prost- |
| # | Structure | 1 | 2 | 3 | 4 | 6 | Ac | TT | EC | tion | colon | lung | ate |
| 242 9 | 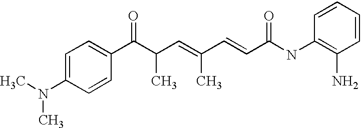 | 3 | 25 | 21 | 23 | >50 | 1 | 1 | 2 | 3 | | | |
| 365 0 | 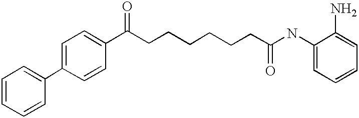 | | 4 | >20 | 23 | >50 | 10 | 5 | 9 | 10 | 53 (40, i p) | | |
| 376 3 | 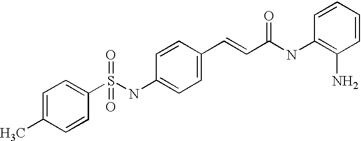 | 3 | 22 | 45 | 28 | >50 | 5 | 4 | 2 | 2 | 55 (40, i p) | | |
| 385 9 | 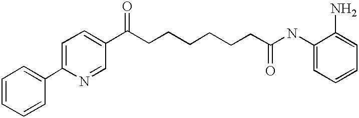 | | 8 | 18 | 13 | >50 | 5 | 5 | 3 | 5 | | | |

note: for in vivo antitumor studies, numbers outside brackets indicate % of inhibition of tumor formation in vivo;
numbers in brackets indicate daily dose of inhibitor used (mg/kg body weight/day);
oral (PO) or intraperitoneal (IP) administration is indicated in brackets.

Small molecule inhibitors of the invention of the formulae Cy-CH(OMe)—Y¹—C(O)—NH-Z, Cy-Y²—C(O)NH-Z and Cy-B—Y³—C(O)—NH-Z, which may be conveniently prepared according to the following schemes 1-3 or using other art-recognized methods.

### Scheme 1

A dialkyl acetal I is treated with 1-trimethylsilyloxy-1,3-butadiene or with 1-trimethylsilyloxy-2,4-dimethyl-1,3-butadiene in the presence of zinc bromide to yield the aldehyde II. Wittig reaction of II with a carboalkoxy phosphorous yield such as ethyl (triphenylphosphoranylidene)acetate yields the diene ester III. Hydrolysis of the ester function in III can be effected by treatment with a hydroxide base, such as lithium hydroxide, to yield the corresponding acid IV.

The acid IV is converted to the corresponding acid chloride V according to standard methods, e.g., by treatment with sodium hydride and oxalyl chloride. Treatment of V with 1,2-phenylenediamine and a tertiary base such as N-methyl-morpholine, preferably in dichloromethane at reduced temperature, then yields the anilinylamide VI. Alternatively, the acid chloride V may be treated with a mono-protected

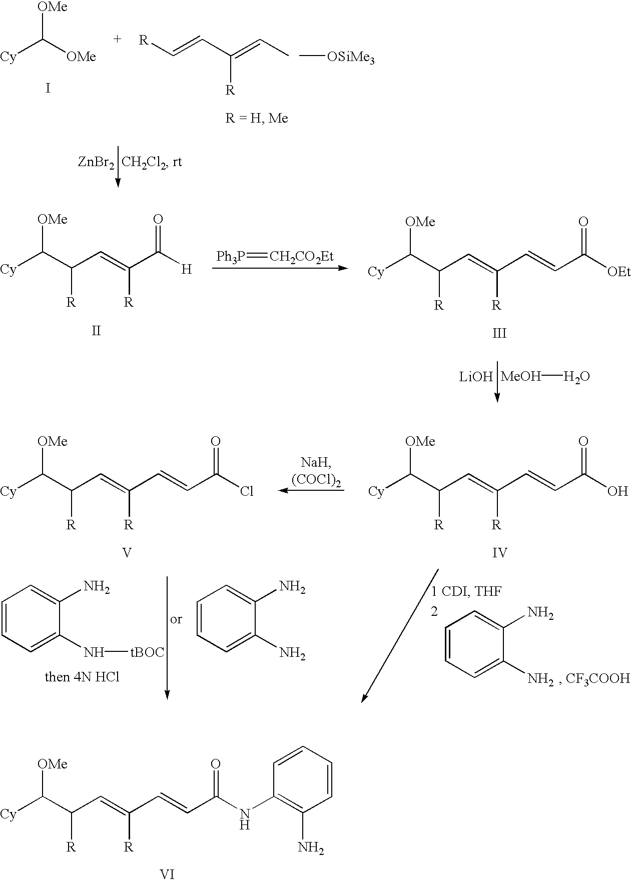

1,2-phenylenediamine, such as 2-(t-BOC-amino)aniline, followed by deprotection, to yield VI.

In an alternative procedure, the acid IV may be activated by treatment with carbonyldiimidazole (CDI), followed by treatment with 1,2-phenylenediamine and trifluoroacetic acid, to yield the anilinyl amide VI.

Compounds of formula Cy-y²-C(O)—NH₂ (VII), wherein Y² is:

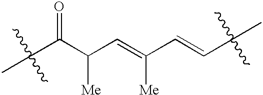

may be prepared from the corresponding methoxy-substituted compounds (VI) by oxidation with 2,3-dichloro-5,6-dicyano-1,4-benzoquinone (DDQ), as illustrated in

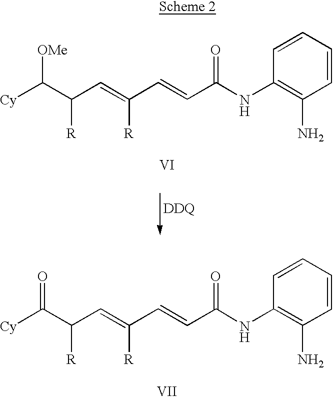

Compounds of formula Cy-y²-C(O)—NH₂, wherein y² has the structure

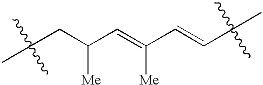

may be prepared as shown in Scheme 3. The methoxy substituted diene ester III, prepared as described in Scheme 1, is treated with triethylsilane and boron trifluoride etherate to yield the deoxygenated compound VIII. Conversion of VIII to the anilinylamide X is accomplished by procedures analogous to those described in Scheme 1.

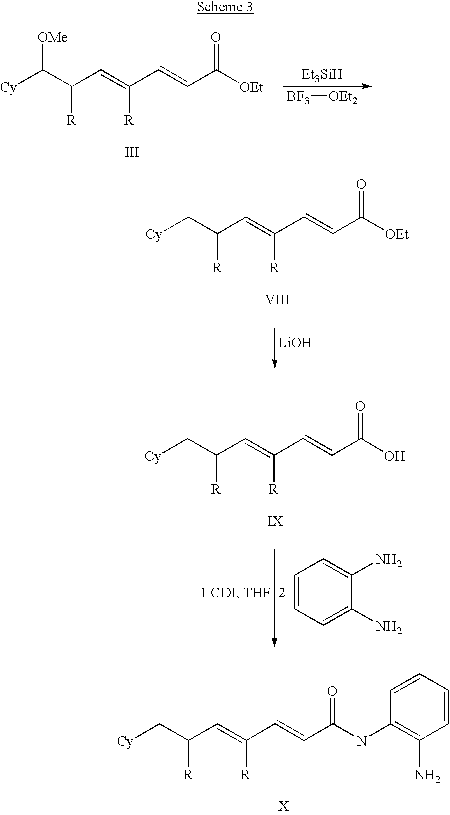

Compounds of formula Cy-L¹-Ar—Y¹—C(O)—NH—O-M, wherein L¹ is —S(O)₂NH—, may be prepared according to the synthetic routes depicted in Schemes 4-6. In certain preferred embodiments, compounds XI are preferably prepared according to the general synthetic route depicted in Scheme 4. A sulfonyl chloride (XII) is treated with an amine (XIII) in a solvent such as methylene chloride in the presence of an organic base such as triethylamine. Treatment of the crude product with a base such as sodium methoxide in an alcoholic solvent such as methanol effects cleavage of any dialkylated material and affords the sulfonamide (XIV). Hydrolysis of the ester function in XIV can be effected by treatment with a hydroxide base, such as lithium hydroxide, in a solvent mixture such as tetrahydrofuran and methanol to yield the corresponding acid (XV).

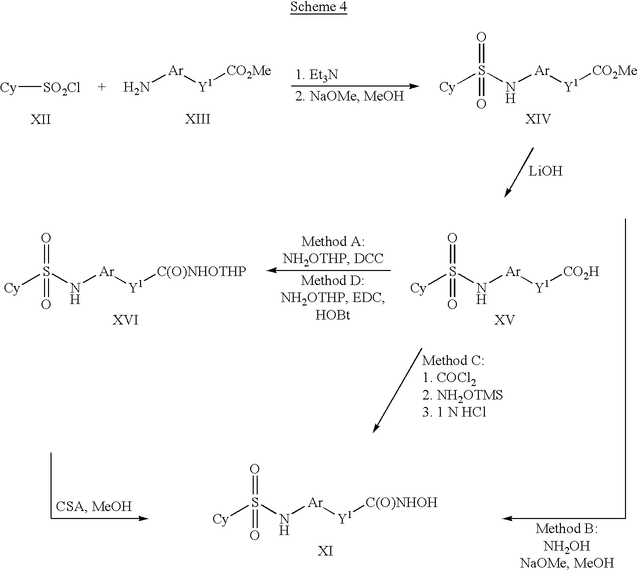

In some embodiments, conversion of the acid XV to the hydroxamic acid XI is accomplished by coupling XV with a protected hydroxylamine, such as tetrahydropyranylhydroxylamine (NH₂OTHP), to yield the protected hydroxamate XVI, followed by acidic hydrolysis of XVI to provide the hydroxamic acid XI. The coupling reaction is preferably accomplished with the coupling reagent dicyclohexylcarbodiimide (DCC) in a solvent such as methylene chloride (Method A), or with the coupling reagent 1-(3-dimethylaminopropyl)-3-ethylcarbodiimide in presence of N-hydroxy benzotriazole in an aprotic solvent such as dimethylformamide (Method D). Other coupling reagents are known in the art and may also be used in this reaction. Hydrolysis of XVI is preferably effected by treatment with an organic acid such as camphorsulfonic acid in a protic solvent such as methanol.

Alternatively, in some other embodiments, acid XV is converted to the corresponding acid chloride, preferably by treatment with oxalic chloride, followed by the addition of a protected hydroxylamine such as O-trimethylsilylhydroxylamine in a solvent such as methylene chloride, which then provides the hydroxamic acid XI upon workup (Method C).

In still other embodiments, the ester XIV is treated with hydroxylamine in a solvent such as methanol in the presence of a base such as sodium methoxide to furnish the hydroxamic acid XI directly (Method B).

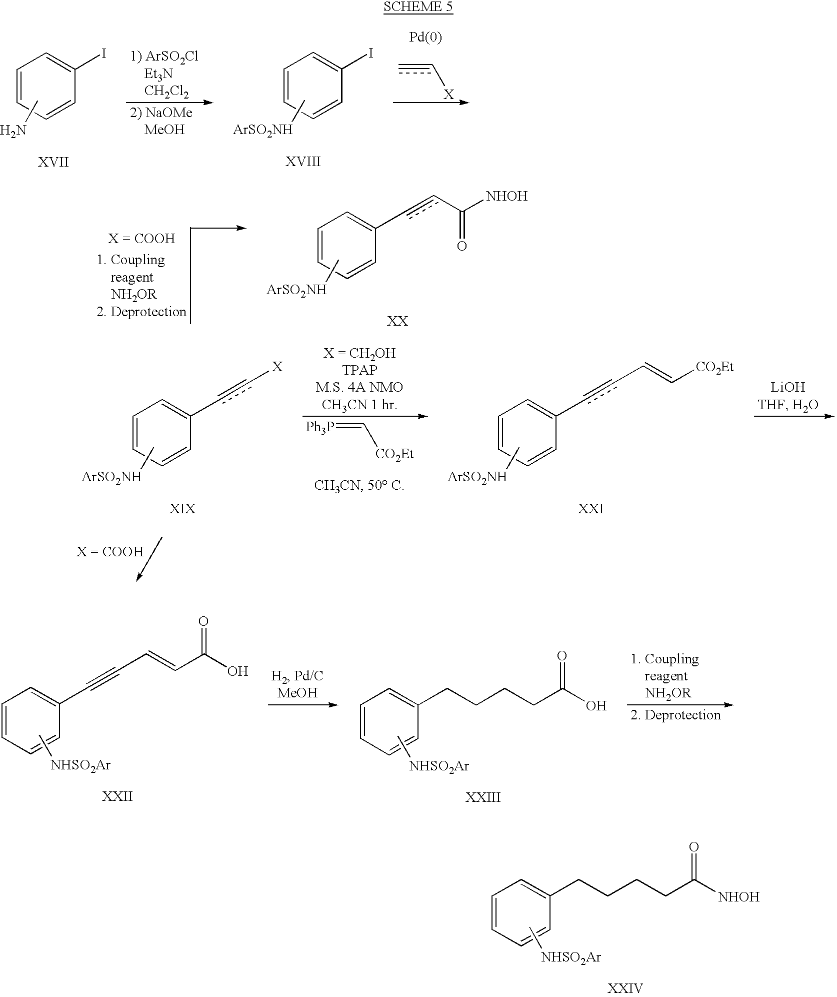

Compounds of formula XX and XXIV preferably are prepared according to the general procedure outlined in Scheme 5 above.

An aminoaryl halide (XVII) is treated with a sulfonyl chloride in presence of a base such as triethylamine, followed by treatment with an alkoxide base, to furnish the sulfonamide XVIII. One of skill in the art will recognize that reverse sulfonamide analogs can be readily prepared by an analogous procedure, treating a haloarenesulfonyl halide with an arylamine.

Compound XVIII is coupled with a terminal acetylene or olefinic compound in the presence of a palladium catalyst such as tetrakis(triphenylphosphine)palladium(0) in a solvent such as pyrrolidine to yield XIX.

Oxidation of the compound of formula XIX (X═CH₂OH), followed by homologation of the resulting aldehyde (using a Wittig type reagent such as carbethoxymethylenetriphenylphosphorane in a solvent such as acetonitrile), yields the compound of formula XXI. Basic hydrolysis of XXI, such as by treatment with lithium hydroxide in a mixture of THF and water, provides the acid XXII. Hydrogenation of XXII may preferably be performed over a palladium catalyst such as Pd/C in a protic solvent such as methanol to yield the saturated acid XXIII. Coupling of the acid XXIII with an O-protected hydroxylamine such as O-tetrahydropyranylhydroxylamine is effected by treatment with a coupling reagent such as 1-(3-dimethylaminopropyl)-3-ethylcarbodiimide in the presence of N-hydroxybenzotriazole (HOBT), or N,N-dicyclohexylcarbodiimide (DCC), in a solvent such as DMF, followed by deprotection to furnish the compound of general formula XXIV.

The acid XIX, wherein X═COOH, may be coupled directly with an O-protected hydroxylamine such as O-tetrahydropyranylhydroxylamine, followed by deprotection of the hydroxy protecting group to furnish the hydroxamic acid XX.

Compounds of formula Cy-L¹-Ar—Y¹—C(O)—NH—O-M, wherein L¹ is —C(O)NH—, preferably may be prepared according to the synthetic routes analogous to those depicted in Schemes 4-5, substituting acid chloride starting materials for the sulfonyl chloride starting materials in those schemes.

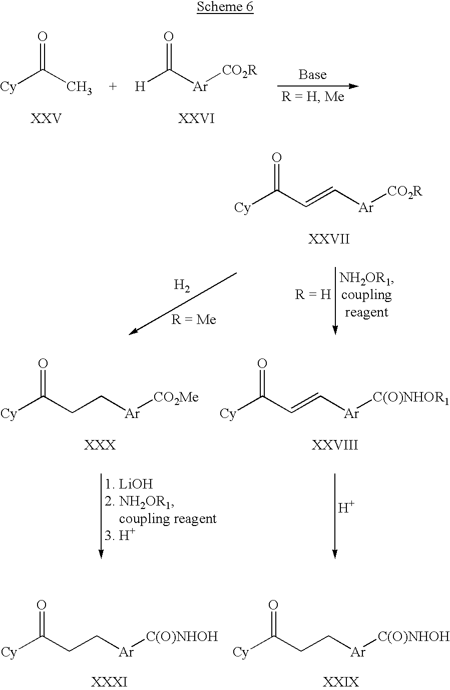

Compounds of the formula Cy-L²-Ar—Y²—C(O)—NH—O-M are preferably prepared according to the synthetic routes outlined in Schemes 6-8. Accordingly, in certain preferred embodiments, compounds of formulae XXIX and XXXI (L²=—C(O)—CH═CH— or —C(O)—CH₂CH₂—) preferably are prepared according to the route described in Scheme 6. Thus, a substituted aryl acetophenone (XXV) is treated with an aryl aldehyde (XXVI) in a protic solvent such as methanol in the presence of a base such as sodium methoxide to afford the enone XXVII.

The acid substituent of XXVII (R═H) is coupled with an O-protected hydroxylamine such as O-tetrahydropyranylhydroxylamine (R₁=tetrahydropyranyl) to yield the O-protected-N-hydroxybenzamide XXVIII. The coupling reaction is preferably performed by treating the acid and hydroxylamine with dicyclohexylcarbodiimide in a solvent such as methylene chloride or with 1-(3-dimethylaminopropyl)-3-ethylcarbodiimide in the presence of N-hydoxybenzotriazole in a solvent such as dimethylformamide. Other coupling reagents are known in the art and may also be used in this reaction. O-Deprotection is accomplished by treatment of XXVIII with an acid such as camphorsulfonic acid in a solvent such as methanol to afford the hydroxamic acid XXIX (L²=—C(O)—CH═CH—).

Saturated compounds of formula XXXI (L²=—C(O)—CH₂CH₂—) are preferably prepared by hydrogenation of XXVII (R=Me) over a palladium catalyst, such as 10% Pd/C, in a solvent such as methanol-tetrahydrofuran. Basic hydrolysis of the resulting saturated ester XXX with lithium hydroxide, followed by N-hydroxy amide formation and acid hydrolysis as described above, then yields the hydroxamic acid XXXI.

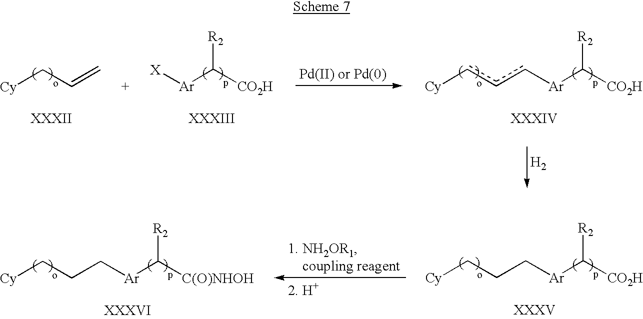

Compounds of formula XXXVI (L²=—(CH₂)ₒ₊₂—) are preferably prepared by the general procedures described in Scheme 7. Thus, in some embodiments, a terminal olefin (XXXII) is coupled with an aryl halide (XXXIII) in the presence of a catalytic amount of a palladium source, such as palladium acetate or tris(dibenzylideneacetone)dipalladium(0), a phosphine, such as triphenylphosphine, and a base, such as triethylamine, in a solvent such as acetonitrile to afford the coupled product XXXIV. Hydrogenation, followed by N-hydroxyamide formation and acid hydrolysis, as described above, yields the hydroxamic acid XXXVI.

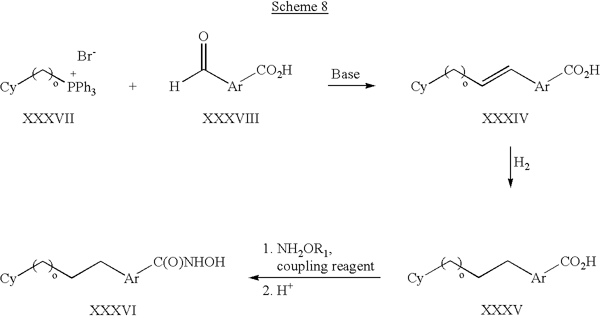

Alternatively, in some other embodiments, a phosphonium salt of formula XXXVII is treated with an aryl aldehyde of formula XXXVIII in the presence of base, such as lithium hexamethyldisilazide, in a solvent, such as tetrahydrofuran, to produce the compound XXXIV. Hydrogenation, followed by N-hydroxyamide formation and acidic hydrolysis, then yields the compounds XXXVI (Scheme 8).

Compounds of formula Cy-L-Ar—Y—C(O)—NH-Z, wherein L is L¹ or L², as previously described herein, Y is Y¹ or Y², as previously described herein, and Z is anilinyl or pyridyl or thiadiazolyl, are preferably prepared according to synthetic routes outlined in Scheme 9.

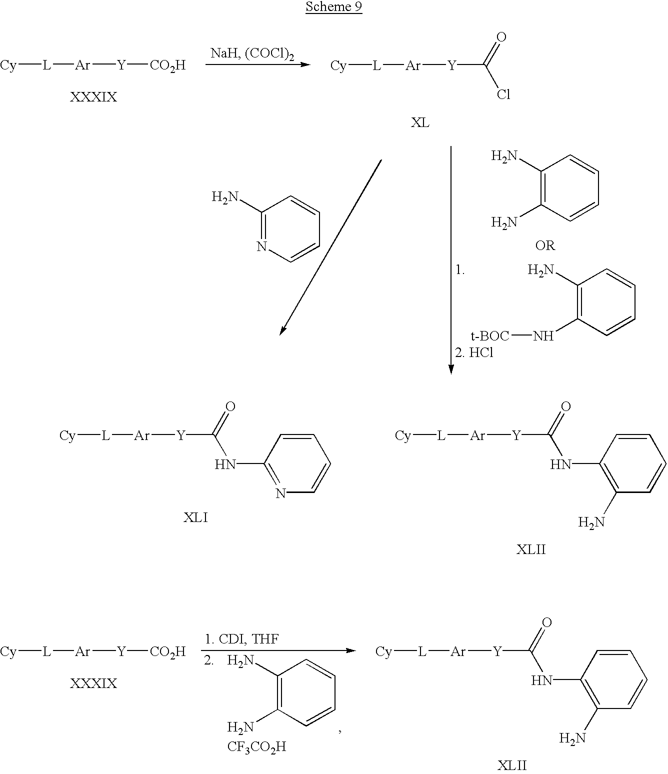

An acid of formula Cy-L-Ar—Y—C(O)—OH (XXXIX), prepared by one of the methods shown in Schemes 4-8, is converted to the corresponding acid chloride XL according to standard methods, e.g., by treatment with sodium hydride and oxalyl chloride. Treatment of XL with 2-aminopyridine and a tertiary base such as N-methylmorpholine, preferably in dichloromethane at reduced temperature, then yields the pyridyl amide XLI. In a similar fashion, the acid chloride XL may be treated with 1,2-phenylenediamine to afford the anilinyl amide XLII. Alternatively, the acid chloride XL may be treated with a mono-protected 1,2-phenylenediamine, such as 2-(t-BOC-amino)aniline, followed by deprotection, to yield XLII.

In an alternative procedure, the acid XXXIX may be activated by treatment with carbonyldiimidazole (CDI), followed by treatment with 1,2-phenylenediamine and trifluoroacetic acid to afford the anilinyl amide XLII.

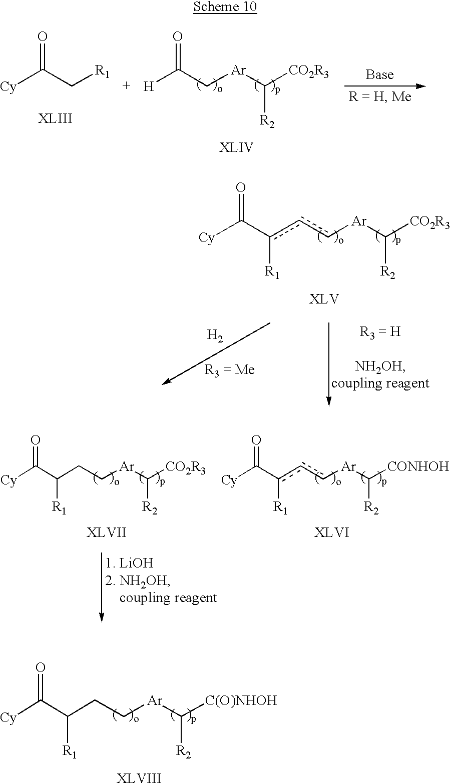

Compounds of formula XLVIII (L²=—C(O)-alkylene-) preferably are prepared according to the general procedure depicted in Scheme 10. Thus, Aldol condensation of ketone XLIII (R₁=H or alkyl) with aldehyde XLIV affords the adduct XLV. The adduct XLV may be directly converted to the corresponding hydroxamic acid XLVI. Hydrogenation of XLV may yield the saturated compound XLVII and which is then converted to the hydroxamic acid XLVIII.

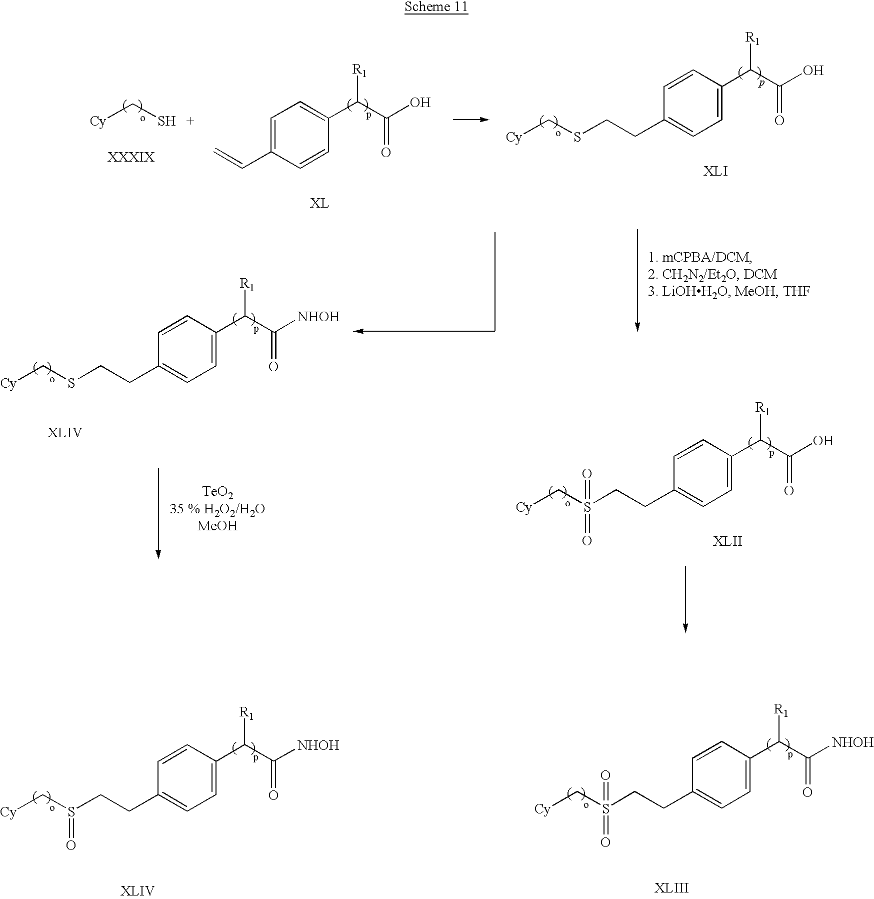

Compounds of formula (5), wherein one of the carbon atoms in L² is replaced with S, S(O), or S(O)₂ preferably are prepared according to the general procedure outlined in Scheme 11. Thus, thiol XLIX is added to olefin L to produce LI. The reaction is preferably conducted in the presence of a radical initiator such as 2,2′-azobisisobutyronitrile (AIBN) or 1,1′-azobis(cyclohexanecarbonitrile) (VAZO™). Sulfide oxidation, preferably by treatment with m-chloroperbenzoic acid (mCPBA), affords the corresponding sulfone, which is conveniently isolated after conversion to the methyl ester by treatment with diazomethane. Ester hydrolysis then affords the acid LII, which is converted to the hydroxamic acid LIII according to any of the procedures described above. The sulfide LI also may be converted directly to the corresponding hydroxamic acid LIV, which then may be selectively oxidized to the sulfoxide LV, for example, by treatment with hydrogen peroxide and tellurium dioxide.

The reagents according to the invention are useful as analytical tools and as therapeutic tools, including gene therapy tools. The invention also provides methods and compositions which may be manipulated and fine-tuned to fit the condition(s) to be treated while producing fewer side effects.

The invention also provides method for inhibiting HDAC-4 activity in a cell, comprising contacting the cell with a specific inhibitor of HDAC-4, whereby HDAC-4 activity is inhibited. As used herein, the term “specific inhibitor” means any molecule or compound that decreases the amount of HDAC RNA, HDAC protein, and/or HDAC protein activity in a cell. Particularly preferred specific inhibitors decrease the amount of RNA, protein, and/or protein activity in a cell for HDAC-1 and/or HDAC-4.

In an embodiment thereof, the invention provides a method for inhibiting the HDAC-4 isoform in a cell comprising contacting the cell with an antisense oligonucleotide of the first aspect of the invention. Preferably, cell proliferation is inhibited in the contacted cell. In preferred embodiments, the cell is a neoplastic cell which may be in an animal, including a human, and which may be in a neoplastic growth. In certain preferred embodiments, the method of the second aspect of the invention further comprises contacting the cell with HDAC-4 small molecule inhibitor that interacts with and reduces the enzymatic activity of the HDAC-4 isoform. In some embodiments, the histone deacetylase small molecule inhibitor is operably associated with the antisense oligonucleotide.

Thus, the antisense oligonucleotides according to the invention are useful in therapeutic approaches to human diseases, including benign and malignant neoplasms, by inhibiting cell proliferation in cells contacted with the antisense oligonucleotides. The phrase “inhibiting cell proliferation” is used to denote an ability of HDAC-4 antisense oligonucleotide or a small molecule HDAC-4 inhibitor (or combination thereof) to retard the growth of cells contacted with the oligonucleotide or small molecule inhibitor, as compared to cells not contacted.

An assessment of cell proliferation can be made by counting cells that have been contacted with the oligonucleotide or small molecule of the invention and compare that number with the number of non-contacted cells using a Coulter Cell Counter (Coulter, Miami, Fla.) or a hemacytometer. Where the cells are in a solid growth (e.g., a solid tumor or organ), such an assessment of cell proliferation can be made by measuring the growth of the tissue or organ with calipers, and comparing the size of the growth of contacted cells with non-contacted cells. Preferably, the term includes a retardation of cell proliferation that is at least 50% of non-contacted cells. More preferably, the term includes a retardation of cell proliferation that is 100% of non-contacted cells (i.e., the contacted cells do not increase in number or size). Most preferably, the term includes a reduction in the number or size of contacted cells, as compared to non-contacted cells. Thus, HDAC-4 antisense oligonucleotide or HDAC-4 small molecule inhibitor that inhibits cell proliferation in a contacted cell may induce the contacted cell to undergo growth retardation, growth arrest, programmed cell death (i.e., to apoptose), or necrotic cell death.

The cell proliferation inhibiting ability of the antisense oligonucleotides according to the invention allows the synchronization of a population of a-synchronously growing cells. For example, the antisense oligonucleotides of the invention may be used to arrest a population of non-neoplastic cells grown in vitro in the G1 or G2 phase of the cell cycle. Such synchronization allows, for example, the identification of gene and/or gene products expressed during the G1 or G2 phase of the cell cycle. Such a synchronization of cultured cells may also be useful for testing the efficacy of a new transfection protocol, where transfection efficiency varies and is dependent upon the particular cell cycle phase of the cell to be transfected. Use of the antisense oligonucleotides of the invention allows the synchronization of a population of cells, thereby aiding detection of enhanced transfection efficiency.

The anti-neoplastic utility of the antisense oligonucleotides according to the invention is described in detail elsewhere in this specification.

In yet other preferred embodiments, the cell contacted with HDAC-4 antisense oligonucleotide is also contacted with HDAC-4 small molecule inhibitor.

As used herein, the term “histone deacetylase small molecule inhibitor” denotes an active moiety capable of interacting with one or more specific histone deacetylase isoforms at the protein level and reducing the activity of that histone deacetylase isoform. Particularly preferred are histone deacteylase small molecule inhibitors that inhibit the HDAC-1 and/or the HDAC-4 isoform. An HDAC-1 small molecule inhibitor is a molecule that reduces the activity of the HDAC-1 isoform. An HDAC-4 small molecule inhibitor is a molecule that reduces the activity of the HDAC-4 isoform. In a preferred embodiment, the reduction of activity is at least 5-fold, more preferably at least 10-fold, most preferably at least 50-fold. In another embodiment, the activity of the histone deacetylase isoform is reduced 100-fold. As discussed below, a preferred histone deacetylase small molecule inhibitor is one that interacts with and reduces the enzymatic activity of HDAC-4 and/or the HDAC-1 isoform that is involved in tumorigenesis.

In a few preferred embodiments, the histone deacetylase small molecule inhibitor is operably associated with the antisense oligonucleotide. As mentioned above, the antisense oligonucleotides according to the invention may optionally be formulated well known pharmaceutically acceptable carriers or diluents. This formulation may further contain one or more one or more additional histone deacetylase antisense oligonucleotide(s), and/or one or more histone deacetylase small molecule inhibitor(s), or it may contain any other pharmacologically active agent.

The term “operably associated with” or “operable association” includes any association between the antisense oligonucleotide and the histone deacetylase small molecule inhibitor which allows an antisense oligonucleotide to inhibit one or more specific histone deacetylase isoform-encoding nucleic acid expression and allows the histone deacetylase small molecule inhibitor to inhibit specific histone deacetylase isoform enzymatic activity. One or more antisense oligonucleotide of the invention may be operably associated with one or more histone deacetylase small molecule inhibitor. In some preferred embodiments, an antisense oligonucleotide of the invention that targets one particular histone deacetylase isoform (e.g., HDAC-4) is operably associated with an small molecule inhibitor which targets the same histone deacetylase isoform (e.g., HDAC-4). A preferred operable association is a hydrolyzable. Preferably, the hydrolyzable association is a covalent linkage between the antisense oligonucleotide and the histone deacetylase small molecule inhibitor. Such a covalent linkage is hydrolyzable, for example, by esterases and/or amidases. Examples of such hydrolyzable associations are well known in the art. Phosphate esters are particularly preferred.

In certain preferred embodiments, the covalent linkage may be directly between the antisense oligonucleotide and the histone deacetylase small molecule inhibitor so as to integrate the histone deacetylase small molecule inhibitor into the backbone of the oligonucleotide. Alternatively, the covalent linkage may be through an extended structure and may be formed by covalently linking the antisense oligonucleotide to the histone deacetylase small molecule inhibitor through coupling of both the antisense oligonucleotide and the histone deacetylase small molecule inhibitor to a carrier molecule such as a carbohydrate, a peptide, a lipid or a glycolipid. Another useful operable associations include lipophilic association, such as the formation of a liposome containing an antisense oligonucleotide and the histone deacetylase small molecule inhibitor covalently linked to a lipophilic molecule. Such lipophilic molecules include, without limitation, phosphotidylcholine, cholesterol, phosphatidylethanolamine, and synthetic neoglycolipids, such as syalyllacNAc-HDPE. In certain preferred embodiments, the operable association may not be a physical association, but simply a simultaneous co-existence in the body, for example, when the antisense oligonucleotide is associated with one liposome and the small molecule inhibitor is associated with another liposome.

In a third aspect, the invention provides a method for inhibiting neoplastic cell proliferation in an animal, comprising administering to an animal having at least one neoplastic cell present in its body a therapeutically effective amount of a specific inhibitor of HDAC-4, whereby neoplastic cell proliferation is inhibited in the animal. In an embodiment thereof, the invention provides a method for inhibiting neoplastic cell growth in an animal. In this method, a therapeutically effective amount of the antisense oligonucleotide of the invention is administered to an animal having at least one neoplastic cell present in its body, the oligonucleotide being administered with a pharmaceutically acceptable carrier for a therapeutically effective period of time. Preferably, the animal is a mammal, particularly a domesticated mammal. Most preferably, the animal is a human.

The term “neoplastic cell” is used to denote a cell that shows aberrant cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of uncharacteristic or untimely cell proliferation that leads to the development of a neoplastic growth.

As used herein, the term “therapeutically effective amount” means the total amount of each active component of the pharmaceutical composition or method that is sufficient to show a meaningful patient benefit, i.e., inhibiting HDAC activity, particularly HDAC-1 and/or HDAC-4 activity or to inhibit neoplastic growth or for the treatment of proliferative diseases and disorders. When applied to an individual active ingredient, administered alone, the term refers to that ingredient alone. When applied to a combination, the term refers to combined amounts of the active ingredients that result in the therapeutic effect, whether administered in combination, serially or simultaneously.

Administration of the synthetic oligonucleotide of the invention used in the pharmaceutical composition or to practice the method of the present invention can be carried out in a variety of conventional ways, such as intraocular, oral ingestion, inhalation, or cutaneous, subcutaneous, intramuscular, or intravenous injection.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% synthetic oligonucleotide and preferably from about 25 to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the synthetic oligonucleotide and preferably from about 1 to 50% synthetic oligonucleotide.

When a therapeutically effective amount of synthetic oligonucleotide of the invention is administered by intravenous, subcutaneous, intramuscular, intraocular, or intraperitoneal injection, the synthetic oligonucleotide will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, subcutaneous, intramuscular, intraperitoneal, or intraocular injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of synthetic oligonucleotide in the pharmaceutical composition of the present invention will depend upon the nature and severity of the condition being treated, and on the nature of prior treatments which the patent has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the various pharmaceutical compositions used to practice the method of the present invention should contain about 10 μg to about 20 mg of synthetic oligonucleotide per kg body or organ weight.

The duration of intravenous therapy using the pharmaceutical composition of the present invention will vary, depending on the severity of the disease being treated and the condition and potential idiosyncratic response of each individual patient. Ultimately the attending physician will decide on the appropriate duration of intravenous therapy using the pharmaceutical composition of the present invention.

In a preferred embodiment, the therapeutic composition of the invention is administered systemically at a sufficient dosage to attain a blood level of antisense oligonucleotide from about 0.01 μM to about 20 μM. In a particularly preferred embodiment, the therapeutic composition is administered at a sufficient dosage to attain a blood level of antisense oligonucleotide from about 0.05 μM to about 15 μM. In a more preferred embodiment, the blood level of antisense oligonucleotide is from about 0.1 μM to about 10 μM.

For localized administration, much lower concentrations than this may be therapeutically effective. Preferably, a total dosage of antisense oligonucleotide will range from about 0.1 mg to about 200 mg oligonucleotide per kg body weight per day. In a more preferred embodiment, a total dosage of antisense oligonucleotide will range from about 1 mg to about 20 mg oligonucleotide per kg body weight per day. In a most preferred embodiment, a total dosage of antisense oligonucleotide will range from about 1 mg to about 10 mg oligonucleotide per kg body weight per day. In a particularly preferred embodiment, the therapeutically effective amount of HDAC-4 antisense oligonucleotide is about 5 mg oligonucleotide per kg body weight per day.

The method may further comprise administering to the animal a therapeutically effective amount of an HDAC-4 small molecule inhibitor with a pharmaceutically acceptable carrier for a therapeutically effective period of time. In some preferred embodiments, the histone deacetylase small molecule inhibitor is operably associated with the antisense oligonucleotide, as described supra.

The histone deacetylase small molecule inhibitor-containing therapeutic composition of the invention is administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor will range from about 0.01 mg to about 100 mg protein effector per kg body weight per day. In a more preferred embodiment, a total dosage of histone deacetylase small molecule inhibitor will range from about 0.1 mg to about 50 mg protein effector per kg body weight per day. In a most preferred embodiment, a total dosage of histone deacetylase small molecule inhibitor will range from about 0.1 mg to about 25 mg protein effector per kg body weight per day. In a particularly preferred embodiment, the therapeutically effective synergistic amount of histone deacetylase small molecule inhibitor (when administered with an antisense oligonucleotide) is about 5 mg per kg body weight per day.

When the method of the invention results in an improved inhibitory effect, the therapeutically effective concentrations of either or both of the nucleic acid level inhibitor (i.e., antisense oligonucleotide) and the protein level inhibitor (i.e., histone deacetylase small molecule inhibitor) required to obtain a given inhibitory effect are reduced as compared to those necessary when either is used individually.

Furthermore, one of skill will appreciate that the therapeutically effective synergistic amount of either the antisense oligonucleotide or the histone deacetylase inhibitor may be lowered or increased by fine tuning and altering the amount of the other component. The invention therefore provides a method to tailor the administration/treatment to the particular exigencies specific to a given animal species or particular patient. Therapeutically effective ranges may be easily determined for example empirically by starting at relatively low amounts and by step-wise increments with concurrent evaluation of inhibition.

In a fourth aspect, the invention provides a method for inhibiting HDAC-4 isoform in a cell comprising contacting the cell with a small molecule inhibitor of the first aspect of the invention. In certain preferred embodiments of the fourth aspect of the invention, cell proliferation is inhibited in the contacted cell. In preferred embodiments, the cell is a neoplastic cell which may be in an animal, including a human, and which may be in a neoplastic growth.

In a fifth aspect, the invention provides a method for inhibiting neoplastic cell growth in an animal comprising administering to an animal having at least one neoplastic cell present in its body a therapeutically effective amount of a small molecule inhibitor of the first aspect of the invention with a pharmaceutically acceptable carrier for a therapeutically effective period of time.

The histone deacetylase small molecule inhibitor-containing therapeutic composition of the invention is administered systemically at a sufficient dosage to attain a blood level histone deacetylase small molecule inhibitor from about 0.01 μM to about 10 μM. In a particularly preferred embodiment, the therapeutic composition is administered at a sufficient dosage to attain a blood level of histone deacetylase small molecule inhibitor from about 0.05 μM to about 10 μM. In a more preferred embodiment, the blood level of histone deacetylase small molecule inhibitor is from about 0.1 μM to about 5 μM. For localized administration, much lower concentrations than this may be effective. Preferably, a total dosage of histone deacetylase small molecule inhibitor ranges from about 0.01 mg to about 100 mg protein effector per kg body weight per day. In a more preferred embodiment, a total dosage of histone deacetylase small molecule inhibitor ranges from about 0.1 mg to about 50 mg protein effector per kg body weight per day. In a most preferred embodiment, a total dosage of histone deacetylase small molecule inhibitor will range from about 0.1 mg to about 25 mg protein effector per kg body weight per day.

In a sixth aspect, the invention provides a method of inhibiting the induction of cell proliferation, comprising contacting a cell with an antisense oligonucleotide that inhibits the expression of HDAC-4 or contacting a cell with a small molecule inhibitor of HDAC-4. In certain preferred embodiments, the cell is a neoplastic cell, and the induction of cell proliferation is tumorigenesis.

The invention further provides for histone deacetylase small molecule inhibitors that may be generated which specifically inhibit the histone deacetylase isoform(s) required for inducing cell proliferation, e.g., HDAC-1 and HDAC-4, while not inhibiting other histone deacetylase isoforms not required for inducing cell proliferation. Accordingly, in a seventh aspect, the invention provides a method for identifying a small molecule histone deacetylase inhibitor that inhibits the HDAC-4 isoform and or the HDAC-1 isoform, which is required for the induction of cell proliferation. The method comprises contacting the HDAC-4 and/or the HDAC-1 isoform with a candidate small molecule inhibitor and measuring the enzymatic activity of the contacted histone deacetylase isoform, wherein a reduction in the enzymatic activity of the contacted histone deacetylase isoform identifies the candidate small molecule inhibitor as a small molecule histone deacetylase inhibitor that inhibits the histone deacetylase isoform, i.e., HDAC-4 and/or HDAC-1.

Measurement of the enzymatic activity of HDAC-4 or HDAC-1 may be achieved using known methodologies. For example, Yoshida et al. (J. Biol. Chem. 265:17174-17179, 1990) describe the assessment of histone deacetylase enzymatic activity by the detection of acetylated histones in trichostatin A treated cells. Taunton et al. (Science 272:408-411, 1996) similarly describes methods to measure histone deacetylase enzymatic activity using endogenous and recombinant HDAC. Both Yoshida et al. (J. Biol. Chem. 265:17174-17179, 1990) and Taunton et al. (Science 272:408-411,1996) are hereby incorporated by reference.

Preferably, the histone deacetylase small molecule inhibitor that inhibits the HDAC-4 and or the HDAC-1 isoform required for induction of cell proliferation is an HDAC-4 small molecule inhibitor that interacts with and reduces the enzymatic activity of the HDAC-4 and/or the HDAC-1 isoform.

In an eighth aspect, the invention provides a method for identifying a small molecule histone deacetylase inhibitor that inhibits the HDAC-4 isoform involved in the induction of cell proliferation. The method comprises contacting a cell with a candidate small molecule inhibitor and measuring the enzymatic activity of the contacted histone deacetylase isoform, wherein a reduction in the enzymatic activity of the HDAC-4 isoform identifies the candidate small molecule inhibitor as a small molecule histone deacetylase inhibitor that inhibits HDAC-4.

In a ninth aspect, the invention provides a small molecule histone deacetylase inhibitor identified by the method of the seventh or the eighth aspects of the invention. Preferably, the histone deacetylase small molecule inhibitor is substantially pure.

In a tenth aspect, the invention provides a method for inhibiting cell proliferation in a cell comprising contacting a cell with at least two reagents selected from the group consisting of an antisense oligonucleotide that inhibits expression of HDAC-4 isoform, a small molecule histone deacetylase inhibitor that inhibits expression or activity of HDAC-4 isoform, an antisense oligonucleotide that inhibits expression of the HDAC-1 isoform, a small molecule histone deacetylase inhibitor that inhibits the expression or the activity of the HDAC-1 isoform, an antisense oligonucleotide that inhibits expression of a DNA methyltransferase, and a small molecule DNA methyltransferase inhibitor. In one embodiment, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

In an eleventh aspect, the invention provides a method of inhibiting neoplastic cell growth comprising contacting a cell with at least two reagents selected from the group consisting of an antisense oligonucleotide that inhibits expression of HDAC-4 isoform, a small molecule histone deacetylase inhibitor that inhibits the expression or the activity of HDAC-4 isoform, an antisense oligonucleotide that inhibits expression of the HDAC-1 isoform, a small molecule histone deacetylase inhibitor that inhibits expression or activity of the HDAC-1 isoform, an antisense oligonucleotide that inhibits expression of a DNA methyltransferase, and a small molecule DNA methyltransferase inhibitor. In one embodiment, the inhibition of cell growth of the contacted cell is greater than the inhibition of cell growth of a cell contacted with only one of the reagents. In certain embodiments, each of the reagents selected from the group is substantially pure. In preferred embodiments, the cell is a neoplastic cell. In yet additional preferred embodiments, the reagents selected from the group are operably associated.

Antisense oligonucleotides that inhibit DNA methyltransferase are described in Szyf and von Hofe, U.S. Pat. No. 5,578,716. DNA methyltransferase small molecule inhibitors include, without limitation, 5-aza-2′-deoxycytidine (5-aza-dC), 5-fluoro-2′-deoxycytidine, 5-aza-cytidine (5-aza-C), or 5,6-dihydro-5-aza-cytidine.

### EXAMPLES

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the appended claims.

### Example 1

### Synthesis and Identification of Antisense Oligonucleotides

Antisense (AS) and mismatch (MM) oligodeoxynucleotides (oligos) were designed to be directed against the 5′- or 3′-untranslated region (UTR) of the targeted gene. Oligos were synthesized with the phosphorothioate backbone and the 4×4 nucleotides 2′-O-methyl modification on an automated synthesizer and purified by preparative reverse-phase HPLC. All oligos used were 20 base pairs in length.

To identify antisense oligodeoxynucleotide (ODN) capable of inhibiting HDAC-1 expression in human cancer cells, eleven phosphorothioate ODNs containing sequences complementary to the 5′ or 3′ UTR of the human HDAC-1 gene (GenBank Accession No. U50079) were initially screened in T24 cells at 100 nM. Cells were harvested after 24 hours of treatment, and HDAC-1 RNA expression was analyzed by Northern blot analysis. This screen identified HDAC-1 AS as an ODN with antisense activity to human HDAC-1. HDAC-1 MM oligo was created as a control; compared to the antisense oligo, it has a 6-base mismatch.

Twenty-four phosphorothioate ODNs containing sequences complementary to the 5′ or 3′ UTR of the human HDAC-2 gene (GenBank Accession No. U31814) were screened as above. HDAC-2 AS was identified as an ODN with antisense activity to human HDAC-2. HDAC-2 MM was created as a control; compared to the antisense oligo, it contains a 7-base mismatch.

Twenty-one phosphorothioate ODNs containing sequences complementary to the 5′ or 3′ UTR of the human HDAC-3 gene (GenBank Accession No. AF039703) were screened as above. HDAC-3 AS was identified as an ODN with antisense activity to human HDAC-3. HDAC-3 MM oligo was created as a control; compared to the antisense oligo, it contains a a 6-base mismatch.

Seventeen phosphorothioate ODNs containing sequences complementary to the 5′ or 3′ UTR of the human HDAC-4 gene (GenBank Accession No. AB006626) were screened as above. HDAC-4 AS was identified as an ODN with antisense activity to human HDAC-4. HDAC-4 MM oligo was created as a control; compared to the antisense oligo, it contains a 6-base mismatch.

Thirteen phosphorothloate ODNs containing sequences complementary to the 5′ or 3′ untranslated regions of the human HDAC-6 gene (GenBank Accession No. AJ011972) were screened as above. HDAC-6 AS was identified as an ODN with antisense activity to human HDAC-6. HDAC-6 MM oligo was created as a control; compared to the antisense oligo, it contains a 7-base mismatch.

### Example 2

### HDAC AS ODNs Specifically Inhibit Expression at the mRNA Level

In order to determine whether AS ODN treatment reduced HDAC expression at the mRNA level, Human A549 cells were treated with 50 nM of antisense (AS) oligo directed against human HDAC-3 or its corresponding mismatch (MM) oligo for 48 hours, and A549 cells were treated with 50 nM or 100 nM of AS oligo directed against human HDAC-4 or its MM oligo (100 nM) for 24 hours.

Briefly, human A549 and/or T24 human bladder carcinoma cells were seeded in 10 cm tissue culture dishes one day prior to oligonucleotide treatment. The cell lines were obtained from the American Type Culture Collection (ATCC) (Manassas, Va.) and were grown under the recommended culture conditions. Before the addition of the oligonucleotides, cells were washed with PBS (phosphate buffered saline). Next, lipofectin transfection reagent (GIBCO BRL Mississauga, Ontario, Calif.), at a concentration of 6.25 μg/ml, was added to serum free OPTIMEM medium (GIBCO BRL, Rockville, Md.), which was then added to the cells. The oligonucleotides to be screened were then added directly to the cells (i.e., one oligonucleotide per plate of cells). Mismatched oligonucleotides were used as controls. The same concentration of oligonucleotide (e.g., 50 nM) was used per plate of cells for each oligonucleotide tested.

Cells were harvested, and total RNAs were analyzed by Northern blot analysis. Briefly, total RNA was extracted using RNeasy miniprep columns (QIAGEN). Ten to twenty μg of total RNA was run on a formaldehyde-containing 1% agarose gel with 0.5 M sodium phosphate (pH 7.0) as the buffer system. RNAs were then transferred to nitrocellulose membranes and hybridized with the indicated radiolabeled DNA probes. Autoradiography was performed using conventional procedures.

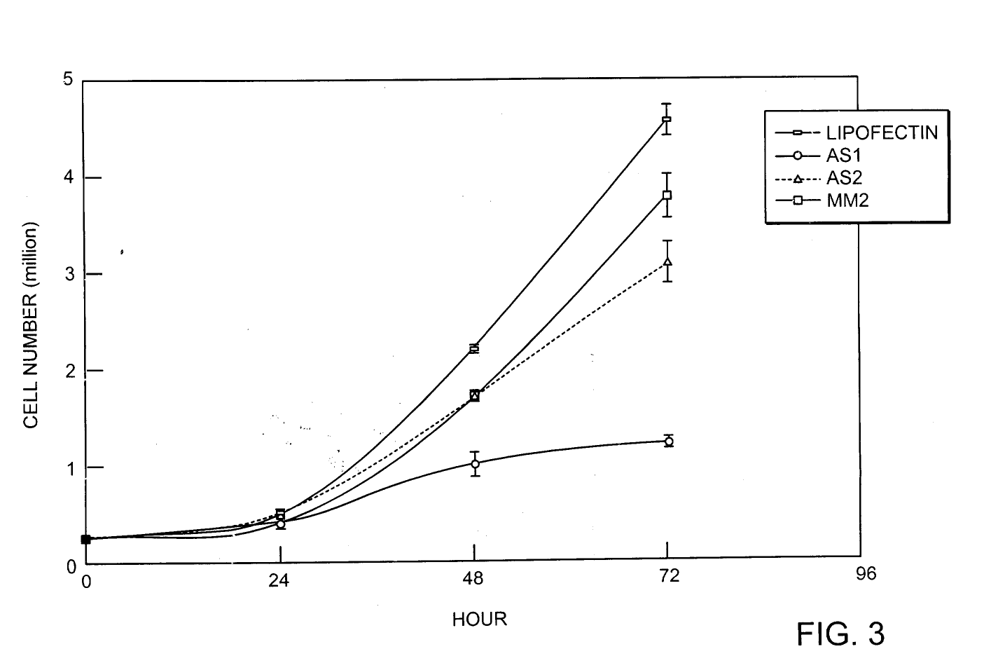

As presented in FIGS. 3A and 3B, respectively, the expression of HDAC-3 mRNA and HDAC-4 mRNA in human A549 cells was inhibited by treatment with the respective antisense oligonucleotides. These results indicate that HDAC AS ODNs can specifically inhibit targeted HDAC expression at the mRNA level.

### Example 3

### HDAC OSDNs Inhibit HDAC Protein Expression

In order to determine whether treatment with HDAC OSDNs would inhibit HDAC protein expression, human A549 cancer cells were treated with 50 nM of paired antisense or its mismatch oligos directed against human HDAC-1, HDAC-2, HDAC-3, HDAC-4 or HDAC-6 for 48 hours. OSDN treatment conditions were as previously described.

Cells were lysed in buffer containing 1% Triton X-100, 0.5% sodium deoxycholate, 5 mM EDTA, 25 mM Tris-HCl, pH 7.5, plus protease inhibitors. Total protein was quantified by the protein assay reagent from Bio-Rad (Hercules, Calif.). 100 ug of total protein was analyzed by SDS-PAGE. Next, total protein was transferred onto a PVDF membrane and probed with various HDAC-specific primary antibodies. Rabbit anti-HDAC-1 (H-51), anti-HDAC-2 (H-54) antibodies (Santa Cruz Biotechnologies, Santa Cruz, Calif.) were used at 1:500 dilution. Rabbit anti-HDAC-3 antibody (Sigma, St. Louis, Mo.) was used at a dilution of 1:1000. Anti-HDAC-4 antibody was prepared as previously described (Wang, S. H. et al., (1999) Mol. Cell. Biol. 19:7816-27), and was used at a dilution of 1:1000. Anti-HDAC-6 antibody was raised by immunizing rabbits with a GST fusion protein containing a fragment of HDAC-6 protein (amino acid #990 to #1216, GenBank Accession No. AAD29048). Rabbit antiserum was tested and found only to react specifically to the human HDAC-6 isoform. HDAC-6 antiserum was used at 1:500 dilution in Western blots to detect HDAC-6 in total cell lysates. Horse Radish Peroxidase conjugated secondary antibody was used at a dilution of 1:5000 to detect primary antibody binding. The secondary antibody binding was visualized by use of the Enhanced chemiluminescence (ECL) detection kit (Amersham-Pharmacia Biotech., Inc., Piscataway, N.J.).

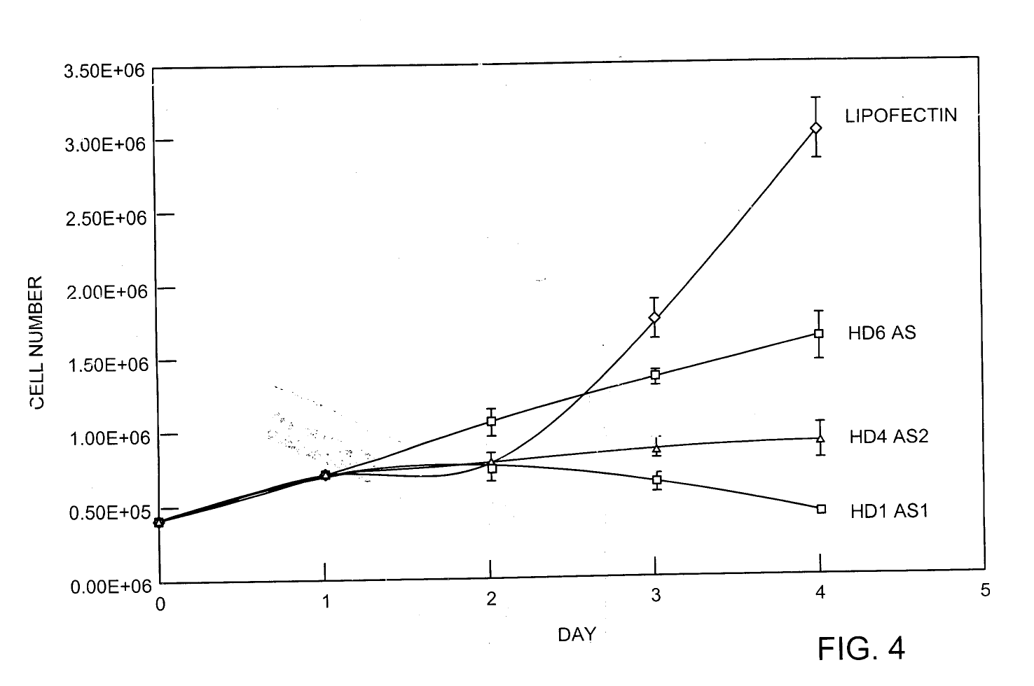

As shown in FIG. 4, the treatment of cells with HDAC-1, HDAC-2, HDAC-3, HDAC-4 or HDAC-6 ODNs for 48 hours specifically inhibits the expression of the respective HDAC isotype protein.

In order to demonstrate that the level of HDAC protein expression is an important factor in the cancer cell phenotype, experiments were done to determine the level of HDAC isotype expression in normal and cancer cells. Western blot analysis was performed as described above.

The results are presented in Table 3 clearly demonstrate that HDAC-1, HDAC-2, HDAC-3, HDAC-4, and HDAC-6, isotype proteins are overexpressed in cancer cell lines.

TABLE 3
Expression Level of HDAC Isotypes in Human Normal and Cancer Cells

| Normal | | | | | | | | | |
|---|---|---|---|---|---|---|---|---|---|
| or | Tissue | Cell | | | | | | | |
| Cancer | Type | Designation | HDAC-6 | HDAC-2 | HDAC-1 | HDAC-3 | HDAC-4 | HDAC-5 | HDAC-7 |
| Normal | Breast | HMEC | + | + | − | ++ | + | − | − |
| | Epithelial | | | | | | | | |
| Normal | Foreskin | MRHF | + | + | − | + | ++ | − | ++ |
| | Fibroblasts | | | | | | | | |
| Cancer | Bladder | T24 | +++ | ++ | +++ | +++ | ++ | + | ++ |
| Cancer | Lung | A549 | ++ | +++ | ++ | +++ | +++ | +++ | + |
| Cancer | Colon | SW48 | +++ | +++ | +++ | +++ | +++ | | |
| Cancer | Colon | HCT116 | +++ | +++ | ++++ | +++ | ++++ | + | − |
| Cancer | Colon | HT29 | +++ | +++ | +++ | +++ | +++ | | |
| Cancer | Colon | NCI-H446 | ++ | ++++ | ++ | +++ | ++++ | ++++ | ++ |
| Cancer | Cervix | Hela | +++ | ++++ | +++ | +++ | +++ | | |
| Cancer | Prostate | DU145 | +++ | +++ | +++ | +++ | ++++ | | |
| Cancer | Breast | MDA-MB- | ++++ | +++ | ++ | +++ | +++ | | |
| | | 231 | | | | | | | |
| Cancer | Breast | MCF-7 | ++ | +++ | +++ | +++ | ++ | | |
| Cancer | Breast | T47D | +++ | +++ | +++ | +++ | ++ | | |
| Cancer | Kidney | 293T | ++ | ++++ | +++ | ++++ | ++ | ++++ | + |
| Cancer | Leukemia | K562 | ++++ | ++++ | +++ | ++++ | ++++ | | |
| Cancer | Leukemia | Jurkat T | ++ | ++ | +++ | ++++ | ++ | ++ | + |

(−): not detectable;
(+): detectable;
(++): 2× over (+);
(+++): 5× over (+);
(++++): 10× over (+)

### Example 4

### Effect of HDAC Isotype Specific OSDNs on Cell Growth and Apoptosis

In order to determine the effect of HDAC OSDNs on cell growth and cell death through apoptosis, A549 or T24 cells, MDAmb231 cells, and HMEC cells (ATCC, Manassas, Va.) were treated with HDAC OSDNs as previously described.

For the apoptosis study, cells were analyzed using the Cell Death Detection ELISA Plus kit (Roche Diagnostic GmBH, Mannheim, Germany) according to the manufacturer's directions. Typically, 10,000 cells were plated in 96-well tissue culture dishes for 2 hours before harvest and lysis. Each sample was analyzed in duplicate. ELISA reading was done using a MR700 plate reader (DYNEX Technology, Ashford, Middlesex, England) at 410 nm. The reference was set at 490 nm.

For the cell growth analysis, human cancer or normal cells were treated with 50 nM of paired AS or MM oligos directed against human HDAC-1, HDAC-2, HDAC-3, HDAC-4 or HDAC-6 for 72 hours. Cells were harvested and cell numbers counted by trypan blue exclusion using a hemocytometer. Percentage of inhibition was calculated as (100-AS cell numbers/control cell numbers)%.

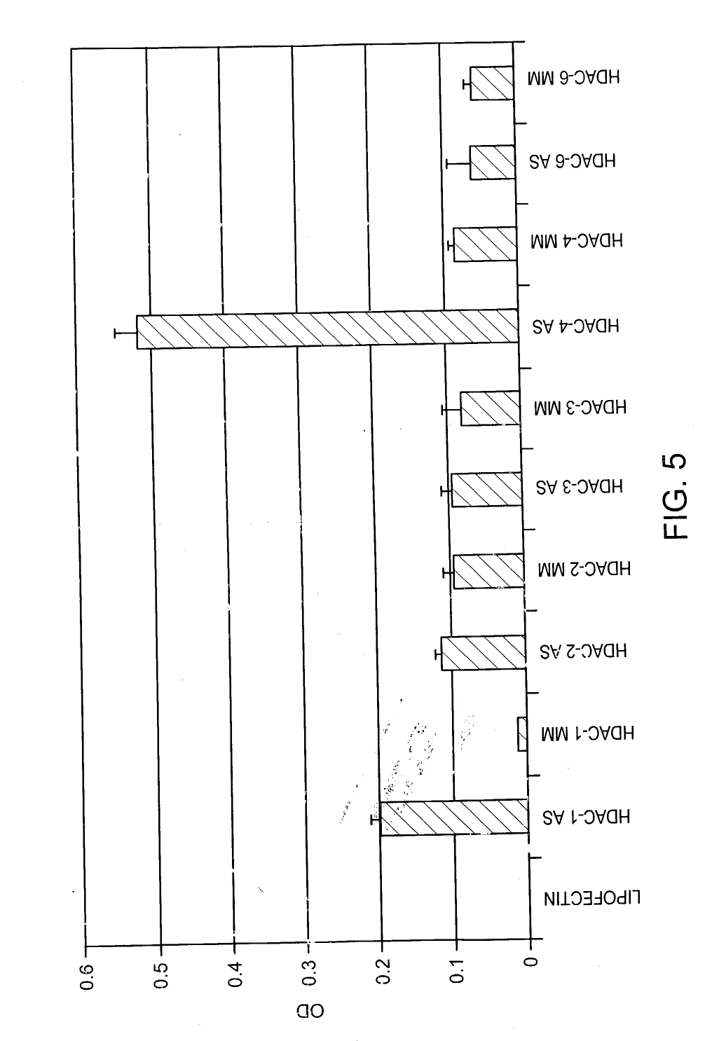

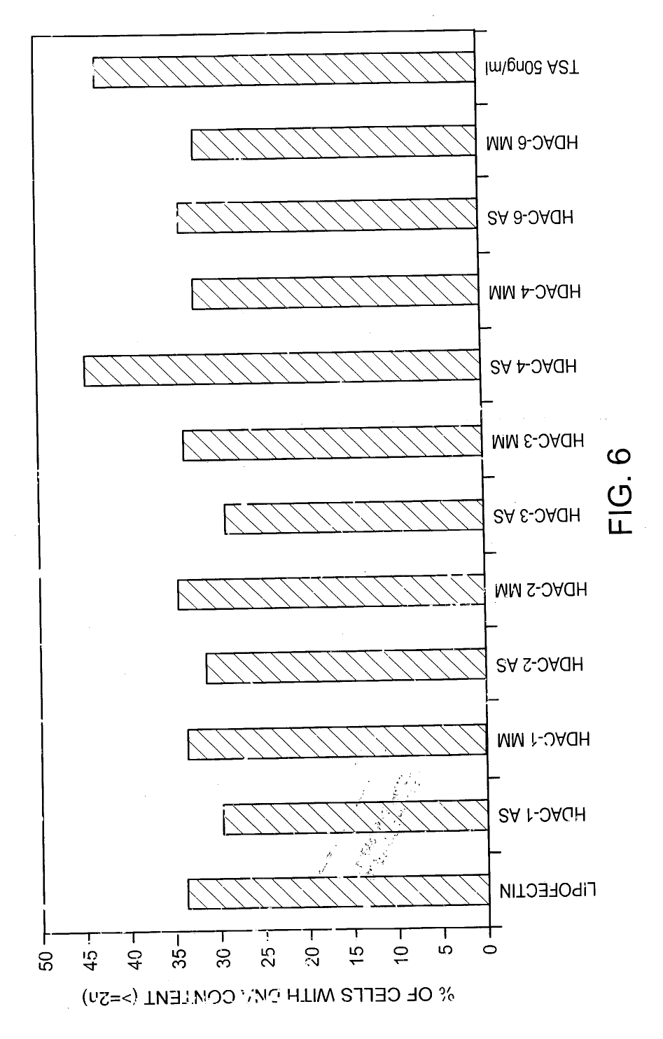

Results of the study are shown in FIG. 5 and FIG. 6, and in Table 4 and Table 5. Treatment of human cancer cells by HDAC-4 AS, and to a lesser extent, HDAC 1 AS, induces growth arrest and apoptosis of various human cancer cells (FIG. 5 and FIG. 6, Table 4 and Table 5). The corresponding mismatches have no effect. The effects of HDAC-4 AS or HDAC-1 AS on growth inhibition and apoptosis are significantly reduced in human normal cells. In contrast to the effects of HDAC-4 or HDAC-1 AS oligos, treatment with human HDAC-3 and HDAC-6 OSDNs has no effect on cancer cell growth or apoptosis, and treatment with human HDAC-2 OSDN has a minimal effect on cancer cell growth inhibition. Since T24 cells are p53 null and A549 cells are p53 wild type, this induction of apoptosis is independent of p53 activity.

TABLE 4
Gene Transcription Altered by HDAC-4 AS1

| gene name | fold change |
|---|---|
| CDK4 | −3 |
| cyclin A2 | −3 |
| cyclin B1 | −3 |
| p21 | 4 |
| PLK | −4 |
| topo II α | −5 |
| GADD153 | 6 |
| GADD45 | 3 |
| Notch-4 | −3 |
| basic FGF | 2 |
| Egr-1 | 3 |
| IL-15 | 4 |
| IRF | 2 |

Human A549 cells were treated with 50 nM oligos for two days before total RNAs were harvested for cDNA array analysis;

Fold change on transcriptions was compared to that of HDAC-4 mismatch oligo (MM2) treated cells;

Expression of 39 genes altered by HDAC-4 AS1 out of 588 genes

TABLE 5
Effect of HDAC Isotype-Specific OSDNs on Human Normal
and Cancer Cells Apoptosis After 48 Hour Treatment

| | A549 | T24 | MDAmb231 | HMEC |
|---|---|---|---|---|
| HDAC-1 | + | − | | − |
| HDAC-2 | − | − | − | − |
| HDAC-3 | − | − | − | − |
| HDAC-4 | +++ | + | ++ | − |
| HDAC-6 | − | − | − | − |
| TSA (100 ng/ml) | ++ | ++ | ++ | + |

“−”: <= 2× fold over non-specific background;
“+”: 2-3× fold;
“++”: 3-5× fold;
“+++”: 5-8× fold;
“++++”: 8× fold

### Example 5

### Inhibition of HDAC Isotypes Induces the Expression of Growth Regulatory Genes

In order to understand the mechanism of growth arrest and apoptosis of cancer cells induced by HDAC-1 or HDAC-4 AS treatment, RNase protection assays were used to analyze the mRNA expression of cell growth regulators (p21 and GADD45) and proapoptotic gene Bax.

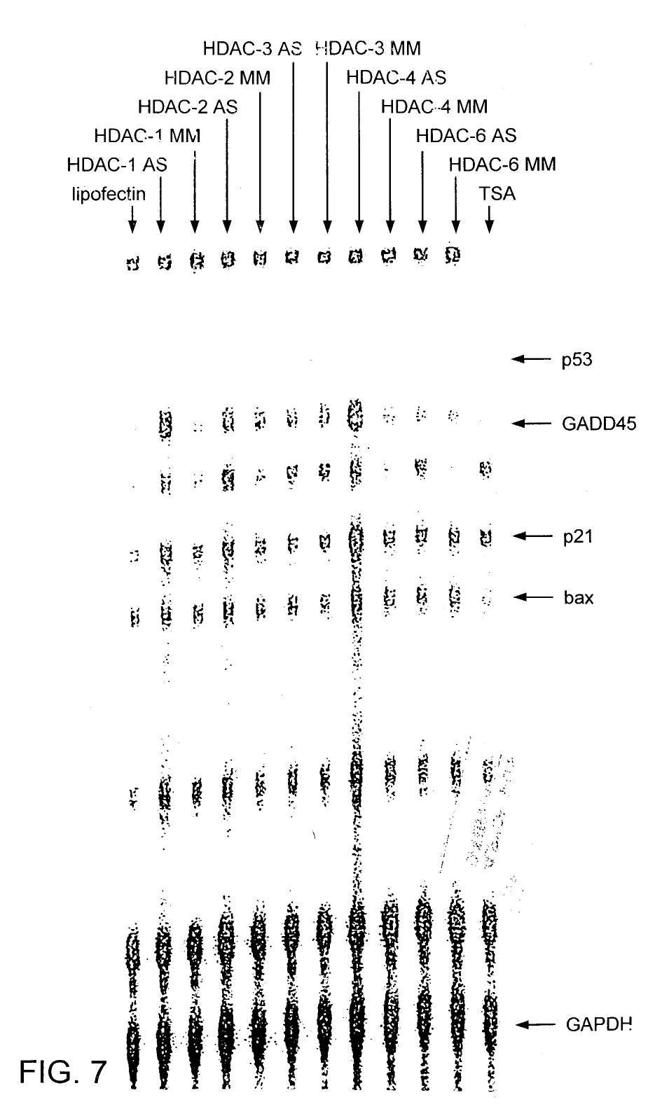

Briefly, human cancer A549 or T24 cells were treated with HDAC isotype-specific antisense oligonucleotides (each 50 nM) for 48 hours. Total RNAs were extracted and RNase protection assays were performed to analyzed the mRNA expression level of p21 and GADD45. As a control, A549 cells were treated by lipofectin with or without TSA (250 ng/ml) treatment for 16 hours. These RNase protection assays were done according to the following procedure. Total RNA from cells was prepared using “RNeasy miniprep kit” from QIAGEN following the manufacturer's manual. Labeled probes used in the protection assays were synthesized using “hStress-1 multiple-probe template sets” from Pharmingen (San Diego, Calif., U.S.A.) according to the manufacturer's instructions. Protection procedures were performed using “RPA II™ Ribonuclease Protection Assay Kit” from Ambion, (Austin, Tx.) following the manufacturer's instructions. Quantitation of the bands from autoradiograms was done by using Cyclone™ Phosphor System (Packard Instruments Co. Inc., Meriden, Conn.). The results are shown in FIG. 7 and Table 6.

TABLE 6
Up-Regulation of p21, GADD45 and Bax After Cell
Treatment with Human HDAC Isotype-Specific Antisenses

| | A549 | | | T24 | | |
|---|---|---|---|---|---|---|
| | p21 | GADD45 | Bax | p21 | GADD45 | Bax |
| HDAC-1 | 1.7 | 5.0 | 0.8 | 2.4 | 3.4 | 0.9 |
| HDAC-2 | 1.1 | 1.2 | 1.0 | 1.0 | 1.0 | 0.9 |
| HDAC-3 | 0.7 | 0.9 | 1.0 | 0.9 | 1.0 | 1.0 |
| HDAC-4 | 3.1 | 5.7 | 2.6 | 2.8 | 2.7 | 1.9 |
| HDAC-6 | 1.0 | 1.0 | 1.0 | 1.0 | 0.8 | 1.1 |
| TSA vs lipofectin | 2.8 | 0.6 | 0.8 | | | |

Values indicate the fold induction of transcription as measured by RNase protection analysis for the respective AS vs. MM HDAC isotype-specific oligos.

As can be seen in FIG. 7, the inhibition of HDAC-4 in both A549 and T24 cancer cells dramatically up-regulates both p2¹ and GADD45 expression. Inhibition of HDAC-1 by antisense oligonucleotides induces p21 expression but more greatly induces GADD45 expression. Inhibition of HDAC-4, upregulates Bax expression in both A549 and T24 cells. The effect of HDAC-4 AS treatment (50 nM, 48 hrs) on p21 induction in A549 cells is comparable to that of TSA (0.3 to 0.8 uM, 16 hrs).

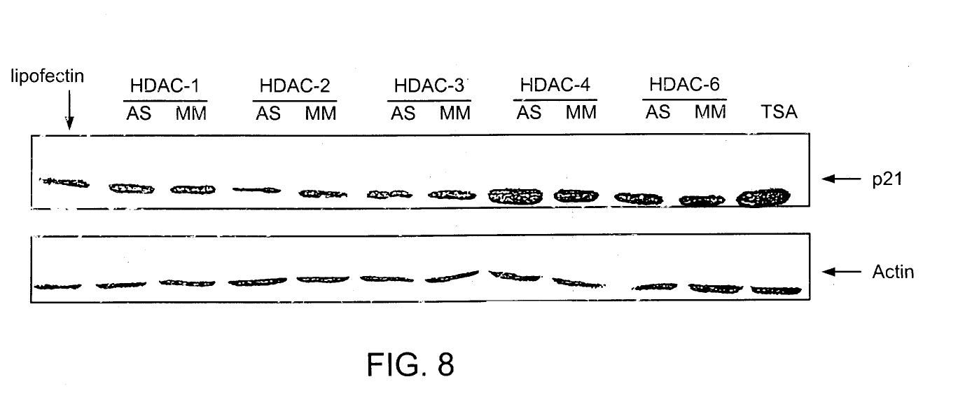

Experiments were also conducted to examine the affect of HDAC antisene oligonucleotides on HDAC protein expression. In A549 cells, treatment with HDAC-4 antisene oligonucleotides results in a dramatic increase in the level of p21 protein (FIG. 8).

### Example 7

### Inhibition of HDAC Isotypes by Small Molecules

In order to demonstrate the identification of HDAC small molecule inhibitors, HDAC small molecule inhibitors were screened in histone deacetylase enzyme assays using various human histone deacetylase isotypic enzymes (i.e., HDAC-1, HDAC-3, HDAC-4 and HDAC-6). Cloned recombinant human HDAC-1, HDAC-3 and HDAC-6 enzymes, which were tagged with the Flag epitope (Grozinger, C. M., et al., Proc. Natl. Acad. Sci. U.S.A. 96:4868-4873 (1999)) in their C-termini, were produced by a baculovirus expression system in insect cells.

Flag-tagged human HDAC-4 enzyme was produced in human embronic kidney 293 cells after transformation by the calcium phosphate precipitation method. Briefly, 293 cells were cultured in Dulbecco's Modified Eagle Medium (DMEM) containing 10% fetal bovine serum and antibiotics. Plasmid DNA encoding Flag-tagged human HDAC-4 was precipitated by ethanol and resuspend in sterile water. DNA-calcium precipitates, formed by mixing DNA, calcium choloride and 2×HEPES-buffered saline solution, were left on 293 cells for 12-16 hours. Cells were return to serum-contained DMEM medium and harvested at 48 hour post transfection for purification of Flag-tagged HDAC-4 enzyme.

HDAC-1 and HDAC-6 were purified on a Q-Sepharose column, followed by an anti-Flag epitope affinity column. The other HDAC isotypes, HDAC-3 and HDAC-4, were purified directly on an anti-Flag affinity column.

For the deacetylase assay, 20,000 cpm of an [³H]-metabolically-labeled acetylated histone was used as a substrate. Histones were incubated with cloned recombinant human HDAC enzymes at 37° C. For the HDAC-1 asasy, the incubation time was 10 minutes, and for the HDAC-3, HDAC-4 and HDAC-6 assays, the incubation time was 2 hours. All assay conditions were pre-determined to be certain that each reaction was linear. Reactions were stopped by adding acetic acid (0.04 M final concentration) and HCl (250 mM, final concentration). The mixture was extracted with ethyl acetate, and the released [3H]-acetic acid was quantified by liquid scintillation counting. For the inhibition studies, HDAC enzyme was preincubated with test compounds for 30 minutes at 4° C. prior to the start of the enzymatic assay. IC₅₀ values for HDAC enzyme inhibitors were identified with dose response curves for each individual compound and, thereby, obtaining a value for the concentration of inhibitor that produced fifty percent of the maximal inhibition.

### Example 8

### Inhibition of HDAC Activity in Whole Cells by Small Molecules

T24 human bladder cancer cells (ATCC, Manassas, Va.) growing in culture were incubated with test compounds for 16 hours. Histones were extracted from the cells by standard procedures (see e.g. Yoshida et al., supra) after the culture period. Twenty μg total core histone protein was loaded onto SDS/PAGE and transferred to nitrocellulose membranes, which were then reacted with polyclonal antibody specific for acetylated histone H-4 (Upstate Biotech Inc., Lake Placid, Wyo.). Horse Radish Peroxidase conjugated secondary antibody was used at a dilution of 1:5000 to detect primary antibody binding. The secondary antibody binding was visualized by use of the Enhanced chemiluminescence (ECL) detection kit (Amersham-Pharmacia Biotech., Inc., Piscataway, N.J.). After exposure to film, acetylated H-4 signal was quantitated by densitometry.

The results, shown in Table 2 above, demonstrate that small molecule inhibitors selective for HDAC-1 and/or HDAC-4 can inhibit histone deacetylation in whole cells.

### Example 9

### Inhibition of Cancer Growth by HDAC Small Molecule Inhibitors

Four thousand five hundred (4,500) human colon cancer HCT116 cells (ATCC, Manassas, Va. were used in an MTT (3-[4,5-dimethylthiazol-2-yl]-2,5 diphenyl tetrazolium bromide) assay to quantitatively determine cell proliferation and cytotoxicity. Typically, HCT116 cells were plated into each well of the 96-well tissue culture plate and left overnight to attach to the plate. Compounds at various concentrations (1 uM, 5 uM and 25 uM, in DMSO) were added in triplicate into the culture media (final DMSO concentration 1%) and incubated for 48 hours. MTT solution (obtained from Sigma as powder) was added and incubated with the cells for 4 hours at 37° C. in incubator with 5% CO₂. During the incubation, viable cells convert MTT to a water-insoluble formazan dye. Solubilizing buffer (50% N,N-dimethylformamide, 20% SDS, pH 4.7) was added to cells and incubate for overnight at 37C in incubator with 5% CO₂. Solubilized dye was quantitated by colorimetric reading at 570 nM using a reference of 630 nM.

The results, shown in Table 2 above, demonstrate that small molecule inhibitors selective for HDAC-1 and/or HDAC-4 can affect cell proliferation.

### Example 10

### Inhibition by Small Molecules of Tumor Growth in a Mouse Model

Female BALB/c nude mice were obtained from Charles River Laboratories (Charles River, NY) and used at age 8-10 weeks. Human prostate tumor cells (DU145, 2×10⁶) or human colon cancer cells (HCT116; 2×10⁶) or small lung core A549 2×10⁶ were injected subcutaneously in the animal's flank and allowed to form solid tumors. Tumor fragments were serially passaged a minimum of three times, then approximately 30 mg tumor fragments were implanted subcutaneously through a small surgical incision under general anaesthesia. Small molecule inhibitor administration by intraperotineal or oral administration was initiated when the tumors reached a volume of 100 mm³. For intraperotineal administration, small molecule inhibitors of HDAC (40-50 mg/kg body weight/day) were dissolved in 100% DMSO and administered daily intraperitoneally by injection. For oral administration, small molecule inhibitors of HDAC (40-50 mg/kg body weight/days) were dissolved in a solution containing 65% polyethylene glycol 400 (PEG 400 (Sigma-Aldridge, Mississauga, Ontario, Calif., Catalogue No. P-3265), 5% ethanol, and 30% water. Tumor volumes were monitored twice weekly up to 20 days. Each experimental group contained at least 6-8 animals. Percentage inhibition was calculated using volume of tumor from vehicle-treated mice as controls.

The results, shown in Table 2 above, demonstrate that small molecule inhibitors selective for HDAC-1 and/or HDAC-4 can inhibit the growth of tumor cells in vivo.

### EQUIVALENTS

Those skilled in the art will recognize, or be able to ascertain, using no more than routine experimentation, many equivalents to the specific embodi,emts of the invention described herein. Such equivalents are intended to be encompasssed by the following claims.

What is claimed is:

1. A method of inhibiting HDAC-4 activity in a cell, comprising contacting the cell with an antisense oligonucleotide complementary to a region of RNA that encodes a portion of HDAC-4, whereby HDAC-4 activity is inhibited.

2. The method according to claim 1, wherein the cell is contacted with an HDAC-4 antisense oligonucleotide that is a chimeric oligonucleotide.

3. The method according to claim 1, wherein the cell is contacted with an HDAC-4 antisense oligonucleotide that is a hybrid oligonucleotide.

4. The method according to claim 1, wherein the antisense oligonucleotide has a nucleotide sequence of from about 13 to about 35 nucleotides which is selected from the nucleotide sequence of SEQ ID NO:4.

5. The method according to claim 1, wherein the antisense oligonucleotide has a nucleotide sequence of from about 15 to about 26 nucleotides which is selected from the nucleotide sequence of SEQ ID NO:4.

6. The method according to claim 1, wherein the cell is contacted with an HDAC-4 antisense oligonucleotide that is SEQ ID NO:11.

7. The method according to claim 1, whereby inhibition of HDAC-4 activity in the contacted cell further leads to an inhibition of cell proliferation in the contacted cell.

8. The method according to claim 1, wherein inhibition of HDAC-4 activity in the contacted cell further leads to growth retardation of the contacted cell.

9. The method according to claim 1, wherein inhibition of HDAC-4 activity in the contacted cell further leads to growth arrest of the contacted cell.

10. The method according to claim 1, wherein inhibition of HDAC-4 activity in the contacted cell further leads to programmed cell death of the contacted cell.

11. The method according to claim 8, wherein inhibition of HDAC-4 activity in the contacted cell further leads to necrotic cell death of the contacted cell.

12. A method of inhibiting HDAC-4 activity in a cell, comprising contacting the cell with a small molecule inhibitor of HDAC-4 selected from the group consisting of:

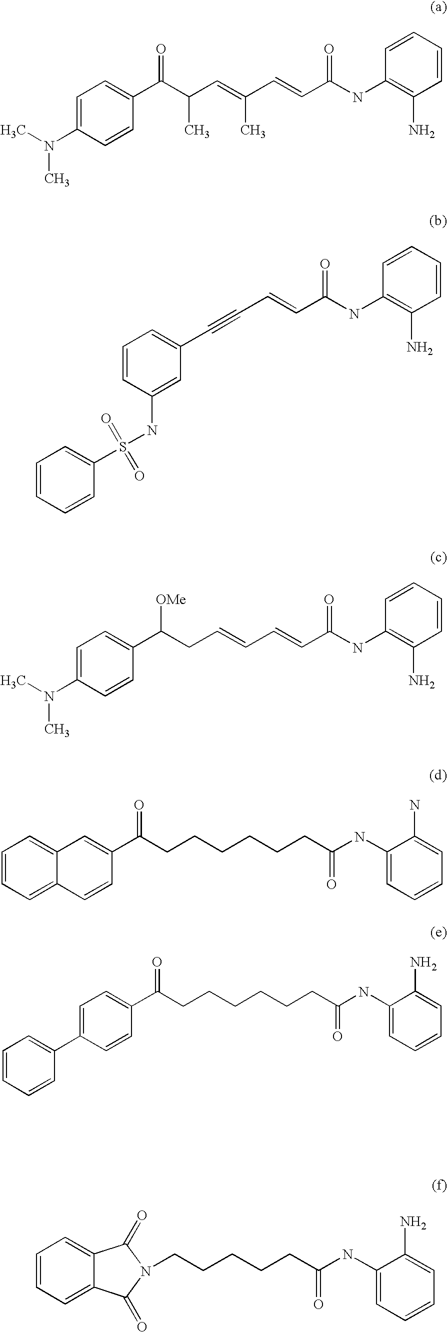

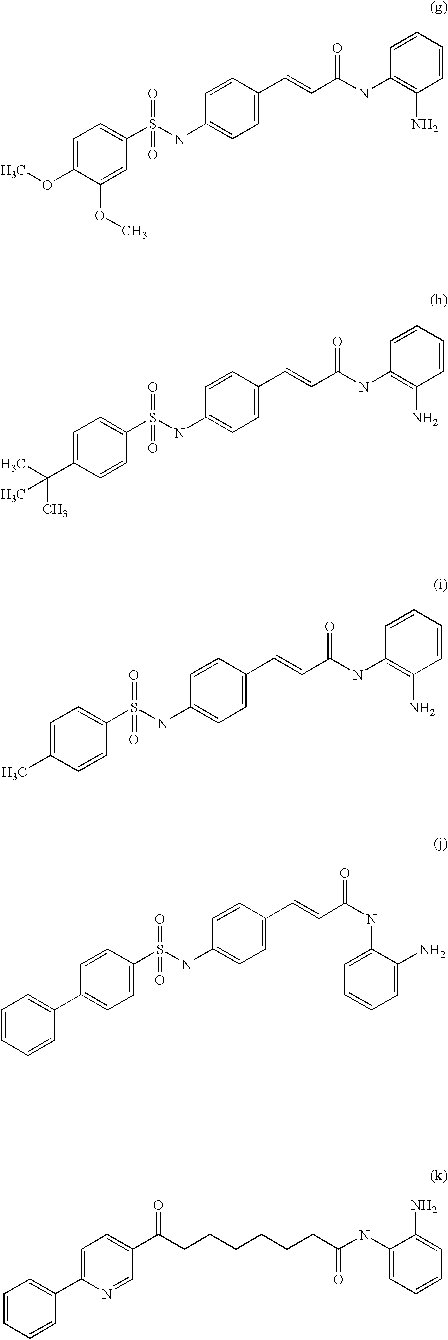

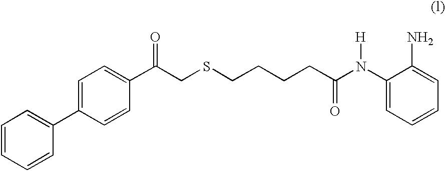

13. The method according to claim 12, whereby inhibition of HDAC-4 activity in the contacted cell further leads to an inhibition of cell proliferation in the contacted cell.

14. The method according to claim 12, wherein inhibition of HDAC-4 activity in the contacted cell further leads to growth retardation of the contacted cell.

15. The method according to claim 12, wherein inhibition of HDAC-4 activity in the contacted cell further leads to growth arrest of the contacted cell.

16. The method according to claim 12, wherein inhibition of HDAC-4 activity in the contacted cell further leads to programmed cell death of the contacted cell.

17. The method according to claim 13, wherein inhibition of HDAC-4 activity in the contacted cell further leads to necrotic cell death of the contacted cell.

18. A method for inhibiting neoplastic cell proliferation in an animal, comprising administering to an animal having at least one neoplastic cell present in its body a therapeutically effective amount of an antisense oligonucleotide complementary to a region of RNA that encodes a portion of HDAC-4, whereby neoplastic cell proliferation is inhibited.

19. The method according to claim 18, wherein the animal is administered a chimeric HDAC-4 antisense oligonucleotide.

20. The method according to claim 18, wherein the animal is administered a hybrid HDAC-4 antisense oligonucleotide.

21. The method according to claim 18, wherein the antisense oligonucleotide has a nucleotide sequence of from about 13 to about 35 nucleotides which is selected from the nucleotide sequence of SEQ ID NO:4.

22. The method according to claim 18, wherein the antisense oligonucleotide has a nucleotide sequence of from about 15 to about 26 nucleotides which is selected from the nucleotide sequence of SEQ ID NO:4.

23. The method according to claim 18, wherein the antisense oligonucleotide has a nucleotide sequence of from about 20 to about 26 nucleotides which is selected from the nucleotide sequence of SEQ ID NO:4.

24. The method according to claim 18, wherein the cell is contacted with an HDAC-4 antisense oligonucleotide that is SEQ ID NO:11.

25. The method according to claim 18, whereby inhibition of HDAC-4 activity in the contacted cell further leads to an inhibition of cell proliferation in the contacted cell.

26. The method according to claim 18, wherein inhibition of HDAC-4 activity in the contacted cell further leads to growth retardation of the contacted cell.

27. The method according to claim 18, wherein inhibition of HDAC-4 activity in the contacted cell further leads to growth arrest of the contacted cell.

28. The method according to claim 18, wherein inhibition of HDAC-4 activity in the contacted cell further leads to programmed cell death of the contacted cell.

29. The method according to claim 25, wherein inhibition of HDAC-4 activity in the contacted cell further leads to necrotic cell death of the contacted cell.

30. A method for inhibiting neoplastic cell proliferation in an animal, comprising administering to an animal having at least one neoplastic cell present in its body a therapeutically effective amount of a small molecule inhibitor selected from the group consisting of:

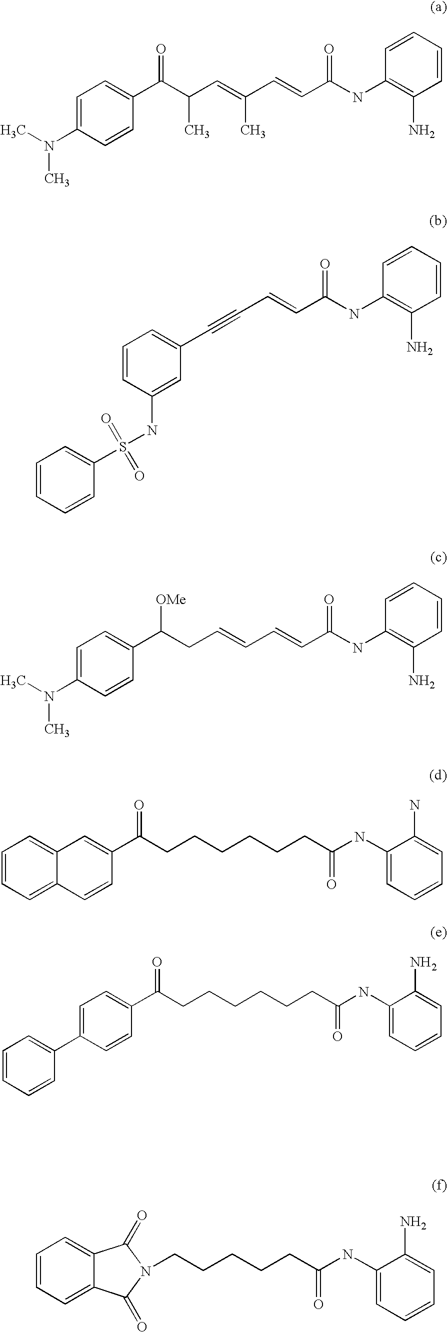

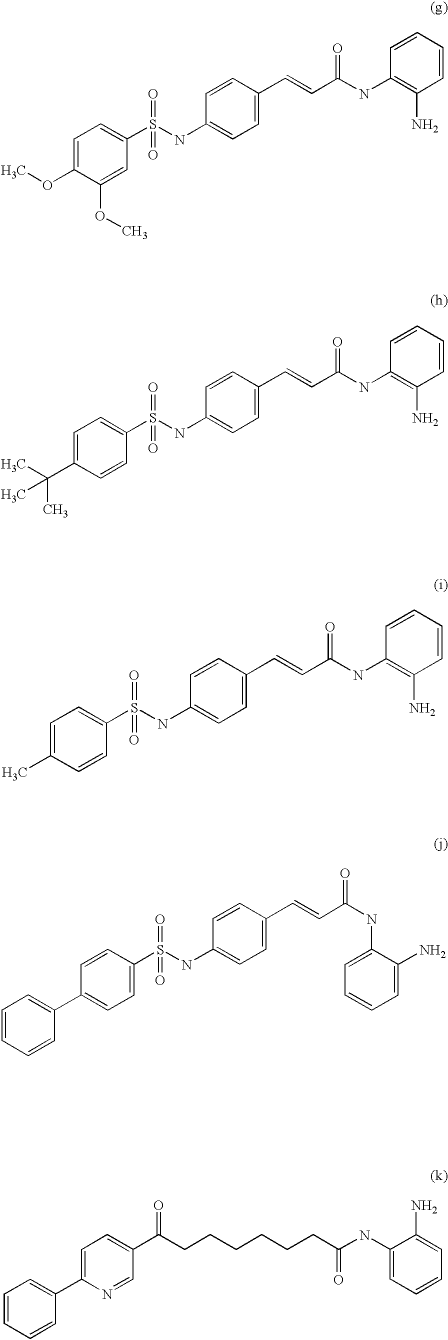

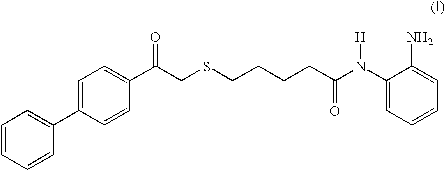

31. The method according to claim 30, whereby inhibition of HDAC-4 activity in the contacted cell further leads to an inhibition of cell proliferation in the contacted cell.

32. The method according to claim 30, wherein inhibition of HDAC-4 activity in the contacted cell further leads to growth retardation of the contacted cell.

33. The method according to claim 30, wherein inhibition of HDAC-4 activity in the contacted cell further leads to growth arrest of the contacted cell.

34. The method according to claim 30, wherein inhibition of HDAC-4 activity in the contacted cell further leads to programmed cell death of the contacted cell.

35. The method according to claim 31, wherein inhibition of HDAC-4 activity in the contacted cell further leads to necrotic cell death of the contacted cell.

36. The method according to claim 18 or 30, wherein the animal is a human.

37. The method according to claim 18 or 30, further comprising administering to an animal a therapeutically effective amount of an antisense oligonucleotide complementary to a region of RNA that encodes a portion of HDAC-1.

38. The method according to claim 37, wherein the animal is administered a chimeric HDAC-1 antisense oligonucleotide.

39. The method according to claim 37, wherein the animal is administered a hybrid HDAC-1 antisense oligonucleotide.

40. The method according to claim 37, wherein the animal is administered an HDAC-1 antisense oligonucleotide having a nucleotide sequence of from about 13 to about 35 nucleotides which is selected from the nucleotide sequence of SEQ ID NO:2.

41. The method according to claim 37, wherein the animal is administered an HDAC-1 antisense oligonucleotide having a nucleotide sequence of from about 15 to about 26 nucleotides which is selected from the nucleotide sequence of SEQ ID NO:2.

42. The method according to claim 37, wherein the animal is administered an HDAC-1 antisense oligonucleotide having a nucleotide sequence of from about 20 to about 26 nucleotides which is selected from the nucleotide sequence of SEQ ID NO:2.

43. The method according to claim 37, wherein the animal is administered an HDAC-1 antisense oligonucleotide that is SEQ ID NO:5.
